(19) United States
(12) Patent Application Publication
(10) Pub. No.: US 2004/0235666 A1
(43) Pub. Date: Nov. 25, 2004

(54) Substituted imidates as pesticidal agents

(76) Inventors: Achim Hense, Sulzbach (DE); Rudiger Fischer, Pulheim (DE); Ernst-Rudolf Gesing, Erkrath (DE); Stefan Herrmann, Langenfeld (DE); Kristian Kather, Langenfeld (DE); Stefan Lehr, Liederbach (DE); Katharina Jansen, Monheim (DE); Hans-Jochem Riebel, Selters (DE); Peter Jeschke, Bergisch Gladbach (DE); Christoph Erdelen, Leichlingen (DE); Angelika Lubos-Erdelen, Leichlingen (DE); Wolfram Andersch, Gladbach (DE); Peter Losel, Leverkusen (DE); Udo Reckmann, Koln (DE)

Correspondence Address: Bayer CropScience, Patent Department, 100 Bayer Road, Pittsburgh, PA, 15205-9741, US

(21) Appl. No.: 10/478,552

(22) PCT Filed: May 16, 2002
(86) PCT No.: PCT/EP02/05389

(30) Foreign Application Priority Data
May 29, 2001 (DE) 10125963.8

(51) Int. Cl. A01N 43/40; A01N 43/36; C07D 211/92; C07D 207/34

(52) U.S. Cl. 504/248; 504/283; 546/223; 548/557

(57) ABSTRACT

The present invention relates to novel substituted imidates of the formula (I)

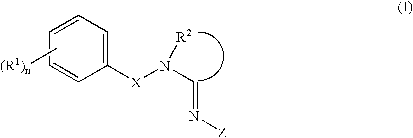

in which Z, n, X, R¹ and R² have the meanings given in the disclosure, and to processes for their preparation, and to their use as pesticides.

The present invention relates to novel substituted imidates, to processes for their preparation and to their use as pesticides.

It is known that certain substituted imidates have insecticidal, acaricidal and/or nematicidal properties (cf., for example, EP-A-235 725, EP-A-386 565 and JP-A-63287764). It is also known that certain substituted imidates are capable of inhibiting phosphodiesterase IV (PDE IV) and cytokines (cf., for example, WO 98/14432).

This invention now provides novel substituted imidates of the formula (I)

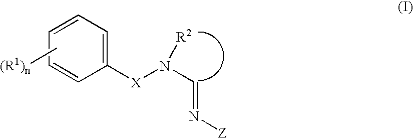

in which

Z represents CN or C(S)NH₂,

X represents substituted methylene or in each case optionally substituted alkylene or alkylidene having in each case 2-6 C atoms, exemplary sub-stituents which may be mentioned being:

in each case optionally halogen-, cyano-, nitro-, C₁-C₄-alkoxy-, C₁-C₄-halo-genoalkoxy-, C₁-C₄-alkylthio- or C₁-C₄-halogenoalkylthio-substituted C₁-C₄-alkyl, C₂-C₄-alkenyl or C₂-C₄-alkinyl;

phenyl which is optionally mono- to trisubstituted by identical or different substituents from the group consisting of halogen, cyano, nitro, C₁-C₄-alkyl, C₁-C₄-halogenoalkyl, C₁-C₄-alkoxy, C₁-C₄-halogenoalkoxy, C₁-C₄-alkylthio and C₁-C₄-halogenoalkylthio,

where two substituents together with the C atom or the C atoms to which they are attached may represent cycloalkyl having 3-6 C atoms,

and where X and R¹ may also be linked by in each case optionally substituted alkylene or alkylidene having 1-3 C atoms, exemplary substituents which may be mentioned being:

in each case optionally halogen-, cyano-, nitro-, C₁-C₄-alkoxy-, C₁-C₄-halogenoalkoxy-, C₁-C₄-alkylthio- or C₁-C₄-halogenoalkylthio-sub-stituted C₁-C₄-alkyl, C₂-C₄-alkenyl or C₂-C₄-alkinyl,

phenyl which is optionally mono- to trisubstituted by identical or different substituents from the group consisting of halogen, cyano, nitro, C₁-C₄-alkyl, C₁-C₄-halogenoalkyl, C₁-C₄-alkoxy, C₁-C₄-halogenoalkoxy, C₁-C₄-alkylthio and C₁-C₄-halogenoalkylthio,

n represents the numbers 1 to 5,

R¹ represents hydroxyl, amino, cyano, nitro, halogen,

represents in each case optionally hydroxyl-, cyano- or halogen-substituted C₁-C₄-alkyl, C₁-C₄-alkoxy, C₁-C₄-alkylamino and di-(C₁-C₄)-alkylamino,

represents in each case optionally halogen-substituted C₁-C₄-alkyl-carbonyl, C₁-C₄-alkoxy-carbonyl, C₁-C₄-alkyl-carbonyl-amino, C₁-C₄-alkylthio, C₁-C₄-alkylsulphinyl and C₁-C₄-alkylsulphonyl, aminocarbonyl, aminothiocarbonyl, C₁-C₄-alkylaminocarbonyl, di-(C₁-C₄)-alkylamino-carbonyl, aminosulphonyl, C₁-C₄-alkylaminosulphonyl and di-(C₁-C₄)-alkylamino-sulphonyl,

represents phenyl, phenoxy, phenylthio, phenylsulphinyl, phenylsulphonyl, benzyl, phenylamino, pyridyloxy or pyridylamino, each of which is optionally substituted by hydroxyl, cyano, nitro, halogen, C₁-C₄-alkyl, C₁-C₄-alkoxy, C₁-C₄-alkylthio, C₁-C₄-alkylsulphinyl, C₁-C₄-alkylsulphonyl and C₁-C₄-halogenoalkyl, C₁-C₄-halogenoalkoxy, C₁-C₄-halogenoalkylthio, C₁-C₄-halogenoalkylsulphinyl and C₁-C₄-halogenoalkylsulphonyl having in each case 1 to 5 identical or different halogen atoms from the group consisting of fluorine, chlorine and bromine,

where two radicals R¹ in the 3- and 4-positions of the phenyl ring do not both represent hydroxyl or optionally substituted alkoxy, phenoxy or pyridyloxy, and

R² together with the N and C atoms to which it is attached represents an optionally C₁-C₆-alkyl-substituted saturated 5- or 6-membered heterocycle having 1 to 3 hetero atoms selected from the group consisting of N, S and O.

The formula (I) provides a general definition of the substituted imidates according to the invention.

Preferred substituents or ranges of the radicals listed in the groupings and formulae mentioned above and below are illustrated below:

X preferably represents substituted methylene, in each case optionally substituted ethylene, propylene or butylene, exemplary substituents which may be mentioned being:

methyl, ethyl, n- or i-propyl, n-, i-, s- or t-butyl, each of which is optionally mono- or polysubstituted by identical or different substituents from the group consisting of fluorine, chlorine, methyl, methoxy and trifluoromethyl,

where two substituents together with the C atom or the C atoms to which they are attached may represent cycloalkyl having 3-6 C atoms,

and where X and R¹ may also be linked by optionally substituted alkylene or alkylidene having 1-3 C atoms, exemplary substituents which may be mentioned being:

methyl, ethyl, n- or i-propyl, n-, i-, s- or t-butyl, each of which is optionally mono- or polysubstituted by identical or different substituents from the group consisting of fluorine, chlorine, methyl, methoxy and trifluoromethyl.

n preferably represents the numbers 1 to 3.

R¹ preferably represents amino, cyano, fluorine, chlorine, bromine, nitro, in each case optionally fluorine- or chlorine-substituted methyl, ethyl, n- or i-propyl, n-, i-, s- or t-butyl, methoxy, ethoxy, n- or i-propoxy, n-, i-, s- or t-butoxy, methylamino, ethylamino, n- or i-propylamino or dimethylamino,

in each case optionally fluorine- and/or chlorine-substituted acetyl, propionyl, methoxycarbonyl, ethoxycarbonyl, n- or i-propoxycarbonyl, methylthio, ethylthio, methylsulphinyl, ethylsulphinyl, methylsulphonyl, ethylsulphonyl, aminocarbonyl, aminothiocarbonyl, methylaminocarbonyl, ethylaminocarbonyl, n- or i-propylamino-carbonyl, dimethylaminocarbonyl, diethylaminocarbonyl, aminosulphonyl, methylaminosulphonyl, ethylaminosulphonyl, dimethylaminosulphonyl and diethylaminosulphonyl,

and also in each case optionally cyano-, nitro-, fluorine-, chlorine-, methyl-, ethyl-, n- or i-propyl-, n-, i-, s- or t-butyl-, trifluoromethyl-, methoxy-, ethoxy-, n- or i-propoxy-, methylthio-, ethylthio-, n- or i-propylthio-, methylsulphinyl-, ethylsulphinyl-, n- or i-propylsulphinyl-, methylsulphonyl-, ethylsulphonyl-, n- or i-propylsulphonyl-, difluoromethoxy-, trifluoromethoxy-, trifluoromethylthio-, trifluoromethylsulphinyl- or trifluoromethylsulphonyl-substituted phenyl, phenoxy, phenylthio, phenylsulphinyl, phenylsulphonyl, benzyl, phenylamino, pyridyloxy or pyridylamino,

where two substituents R¹ in the 3- and 4-positions of the phenyl ring do not both represent optionally fluorine- or chlorine-substituted methoxy, ethoxy, n- or i-propoxy, n-, i-, s- or t-butoxy, or in each case optionally cyano-, nitro-, fluorine-, chlorine, methyl, ethyl, n- or i-propyl-, n-, i-, s- or t-butyl-, trifluoromethyl-, methoxy-, ethoxy-, n- or i-propoxy-, methylthio-, ethylthio-, n- or i-propylthio-, methylsulphinyl-, ethylsulphinyl-, n- or i-propylsulphinyl-, methylsulphonyl-, ethylsulphonyl-, n- or i-propylsulphonyl-, difluoromethoxy-, trifluoromethoxy-, trifluoromethylthio-, trifluoromethylsulphinyl- or trifluoromethylsulphonyl-substituted phenyloxy or pyridyloxy.

R² preferably represents one of the groupings from the group of groupings below:

—CR³₂—CR³₂-A¹-, —CR³₂—CR³₂—CR³₂-A²-, -A³—CR³₂—CR³₂-A⁴-, —CR³₂—CR³₂—NR⁴-A⁵-, —CR³₂—CR³₂—O-A⁶-, —CR³₂—CR³₂—S-A⁷- —CR³₂-A⁸-CR³₂—CR³₂—,

where the first-mentioned atom is attached to the nitrogen in the heterocycle according to formula (I) and where

A¹ represents NR⁴, O , CR³₂, S, SO or SO₂,

A² represents NR⁴, O , CR³₂, S, SO or SO₂,

A³ represents NR⁴ or O,

A⁴ represents NR⁴, O or S,

A⁵ represents NR, O, CR³₂ or S,

A⁶ represents NR⁴, CR³₂ or S,

A⁷ represents CR³₂ or S,

A⁸ represents NR⁴, O, or S,

R³ represents hydrogen or C₁-C₄-alkyl, and

R⁴ represents hydrogen or optionally halogen-, cyano- or C₁-C₆-alkoxy-substituted alkyl having 1-6 C atoms.

X particularly preferably represents substituted methylene or optionally substituted ethylene, exemplary substituents which may be mentioned being: methyl or ethyl, each of which may be mono- or polysubstituted by identical or different substituents from the group consisting of fluorine, chlorine, methyl, methoxy and trifluoromethyl,

where two substituents together with the C atom or the C atoms to which they are attached may represent cycloalkyl having 3-6 C atoms,

and X and R¹ may also be linked by in each case optionally substituted methylene, 1,2-ethanediyl, 1,3-propanediyl, 1,2-ethenediyl or 1,3-propenediyl, exemplary substituents which may be mentioned being:

methyl or ethyl, each of which is optionally mono- or polysubstituted by identical or different substituents from the group consisting of fluorine, chlorine, methyl, methoxy and trifluoromethyl.

n particularly preferably represents the number 1 or 2.

R¹ particularly preferably represents amino, cyano, fluorine, chlorine, bromine, nitro, methyl, ethyl, n- or i-propyl, n-, i-, s- or t-butyl, difluoromethyl, trifluoromethyl, methoxy, ethoxy, n- or i-propoxy, difluoromethoxy, tri-fluoromethoxy, trifluoromethylthio, methylamino, dimethylamino, acetyl, propionyl, methoxycarbonyl, ethoxycarbonyl, methylthio, methylsulphinyl, methylsulphonyl, methylaminocarbonyl, ethylaminocarbonyl, n- or i-propylaminocarbonyl, dimethylaminocarbonyl or diethylaminocarbonyl,

and also in each case optionally amino-, cyano-, fluorine-, chlorine-, bromine-, nitro-, methyl-, trifluoromethyl-, methoxy-, trifluoromethoxy-, methylthio-, methylsulphinyl-, methylsulphonyl-, trifluoromethylthio-, tri-fluoromethylsulphinyl- or trifluoro-methylsulphonyl-substituted phenyl, phenoxy, phenylthio, phenylsulphinyl, phenylsulphonyl, benzyl, phenylamino, pyridyloxy or pyridylamino,

where the substituents R¹ in the 3- and 4-positions of the phenyl ring do not both represent methoxy, ethoxy, n- or i-propoxy, difluoromethoxy, trifluoromethoxy or in each case optionally amino-, cyano-, fluorine-, chlorine-, bromine-, nitro-, methyl-, trifluoromethyl-, methoxy-, trifluoromethoxy-, methylthio-, methylsulphinyl-, methylsulphonyl-, trifluoromethylthio-, trifluoromethylsulphinyl- or trifluoromethylsulphonyl-substituted phenyloxy or pyridyloxy.

R² particularly preferably represents the groupings:

—CH₂—CH₂-A¹-, —CH₂—CH₂—CH₂-A²-, -A³-CH₂—CH₂-A⁴-, —CH₂—CH₂—NR⁴-A⁵-, —CH₂—CH₂—O-A⁶-, —CH₂—CH₂—S-A⁷- —CH₂-A⁸-CH₂—CH₂—,

where the first-mentioned atom is attached to the nitrogen in the heterocycle of the formula (I).

A¹ preferably represents NR⁴, O, CH₂, or S.

A² preferably represents NR⁴, O, S or CH₂.

A³ preferably represents NR⁴ or O.

A⁴ preferably represents NR⁴ or S.

A⁵ preferably represents NR⁴, CH₂ or S.

A⁶ preferably represents NR⁴, CH₂ or S.

A⁷ preferably represents S or CH₂.

A⁸ preferably represents S, O or NR⁴.

R⁴ preferably represents hydrogen or methyl, ethyl, n- or i-propyl, n-, i-, s- or t-butyl, each of which is optionally mono- or polysubstituted by fluorine, chlorine, bromine, cyano, methoxy or ethoxy.

X very particularly preferably represents one of the groupings from the group of groupings below

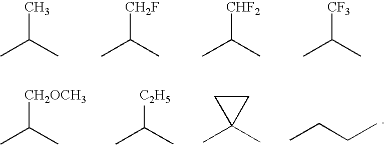

The general or preferred radical definitions or illustrations given above apply both to the end products and, correspondingly, to the starting materials and intermediates. These radical definitions can be combined with one another as desired, i.e. including combinations between the respective preferred ranges.

Preference according to the invention is given to the compounds of the formula (I) which contain a combination of the meanings given above as being preferred (preferable).

Particular preference according to the invention is given to the compounds of the formula (I) which contain a combination of the meanings given above as being particularly preferred.

Very particular preference according to the invention is given to the compounds of the formula (I) which contain a combination of the meanings given above as being very particularly preferred.

In the radical definitions given above and below, hydrocarbon radicals, such as alkyl, are in each case straight-chain or branched as far as this is possible - including in compounds with heteroatoms, such as in alkoxy.

In each case, the formulae given above and below also include the tautomeric forms of these compounds which may optionally be present.

If a plurality of the individual radicals R¹, R³ and R⁴ is attached to the phenyl ring or the heterocycle according to formula (I), these radicals may have identical or different meanings within the scope of the general, preferred, particularly preferred or very particularly preferred definitions given above.

Among the meanings given as being preferred, particularly preferred or very particularly preferred, emphasis has furthermore to be given to the following groups of compounds of the formulae (I-I) (I-II) (I-III), (I-IV), (I-V), (I-VI) and (I-VII):

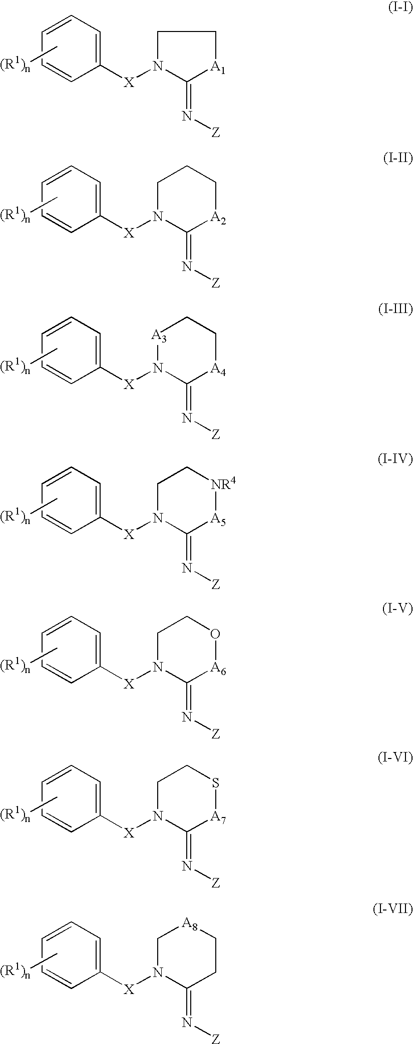

in which

X, Z, n, R¹, R⁴, A¹, A², A³, A⁴, A⁵, A⁶, A⁷ and A⁸ have the general, preferred, particularly preferred and very particularly preferred meanings given above.

Emphasis also is to be given to compounds of the formula (I-c)

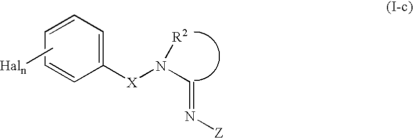

in which

Hal are identical or different and represent F, Cl or Br,

n represents the numbers 1 to 3,

R² represents the groupings:

—CH₂—CH₂-A¹-, —CH₂—CH₂—CH₂-A²-, —CH₂—CH₂—O—CH₂- or —O—CH₂—CH₂—NR⁴—,

where the first-mentioned atom is attached to the nitrogen in the heterocycle of the formula (I-c),

A¹, A² represent NR⁴, O, S or CH₂,

R⁴ represents hydrogen or represents methyl, ethyl, n- or i-propyl, n-, i-, s- or t-butyl, each of which is optionally mono- or polysubstituted by fluorine, chlorine, bromine, cyano, methoxy or ethoxy,

X represents the groupings:

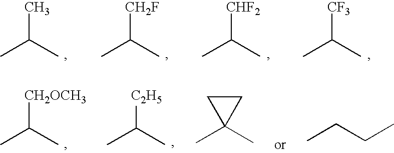

and

Z represents CN or C(S)NH₂.

Furthermore, it has been found that the substituted imidates of the formula (I) are obtained when

a) heterocycles of the formula (II)

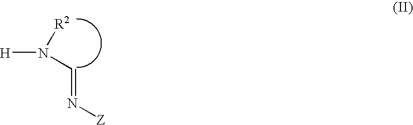

in which

Z represents CN and R² has one of the meanings given above,

also in the form of their salts, preferably the alkali metal salts, such as, in particular, the sodium or potassium salts,

are reacted with substituted phenyl derivatives of the formula (III)

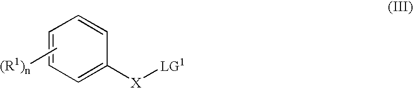

in which

X, R¹ and n have one of the meanings given above and

LG¹ represents a suitable leaving group, such as, for example, halogen, tosylate or sulphonate, preferably chlorine, bromine or tosylate,

or

b) compounds of the formula (IV)

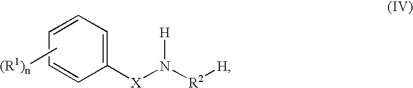

in which

X, R¹, R² and n have one of the meanings given above, where the radicals A¹, A², A⁵, A⁶, A⁷ defined in R² do not represent CR³₂ and where A¹, A², A⁵, A⁶, A⁷ and R³ have the meanings given above,

also in the form of their salts, preferably the alkali metal salts, such as, in particular, the sodium or potassium salts,

are reacted with compounds of the formula (V)

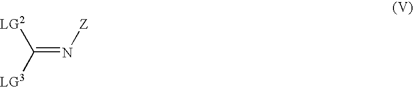

in which

Z represents CN and

LG², LG³ represent a suitable leaving group, such as, for example, aryloxy or alkyl sulphide, preferably phenyloxy or methyl sulphide,

or

c) heterocycles of the formula (VI)

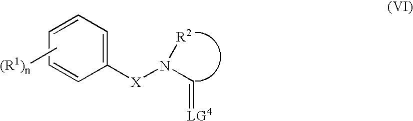

in which X, R¹, R² and n have one of the meanings given above and

LG⁴ represents O or S

are reacted with compounds of the formula (VII)

H₂N-Z (VII)

in which

Z represents CN or C(S)NH₂,

if appropriate in the presence of a diluent and if appropriate in the presence of a basic reaction auxiliary.

The formulae (II) and (III) provide general definitions of the compounds to be used as starting materials for carrying out the process (a) according to the invention. The heterocycles of the formula (II) are known and/or can be prepared as stated in the Preparation Examples. The phenyl derivatives of the formula (III) are known and/or can be prepared analogously to known processes. Thus, the reaction according to process (a) can be carried out analogously to EP 386 565, JP 600 511 84 or JP 631 567 86.

Using, for example, 2-imidazolidinylidenecyanamide and 1-chloro-4-(1-chloroethyl)benzene as starting materials, the course of the reaction in the process (a) according to the invention can be represented by the following reaction scheme:

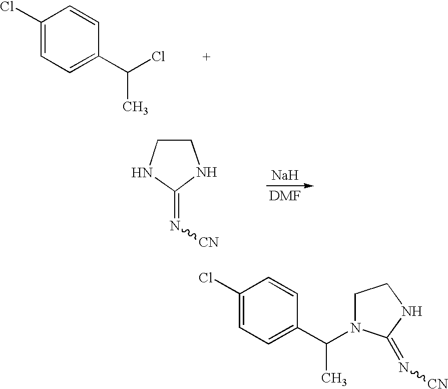

The formulae (IV) and (V) provide general definitions of the compounds used as starting materials for carrying out the process (b) according to the invention. The compounds of formulae (IV) and (V) are known and/or can be prepared analogously to known processes. Thus, the reaction according to process (b) can be carried out, for example, analogously to DE-A-22 057 45.

The formulae (VI) and (VII) provide general definitions of the compounds to be used as starting materials for carrying out the process (c) according to the invention. Some of the heterocycles of the formulae (VI) are already known, and/or can be prepared analogously to known processes. The compounds of the formula (VII) are known.

Thus, the reactions for Z=CN can be carried out analogously to P. W. Manley et al., J. Med. Chem. 1992, 35, 2327; K. Gewald et al., Monatsh. Chem. 1981, 112, 1393 and M. G. Bock et al., J. Med. Chem. 1988, 31, 264, and the reactions for Z=C(S)NH₂ can be carried out analogously to J. Liebscher et al., Z. Chem. 1986, 26, 289 and S. I. Kaimanakova et al., Chem. Heterocycl. Compd. 1982, 18, 1208.

The compounds of the formula (Ia)

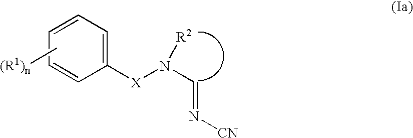

can be converted by methods known to the person skilled in the art into the corresponding compounds (Ib)

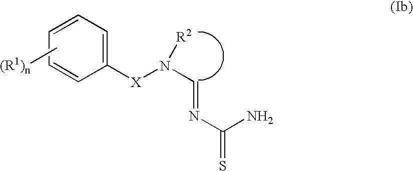

using customary sulphur reagents, such as, for example, hydrogen sulphide or Lawesson's reagent.

The reactions mentioned are described, for example, in DE-A-19523658; EP 0095640; Y. Katsura, et al., Bioorg. Med. Chem. Lett. 1998, 8, 1307 and K.-A. Trumm et al., Arzneim. Forsch. 1985, 35, 573.

Using, for example, 1-[1-(4-chlorophenyl)ethyl]-2-imidazolidinylidenecyanamide and 2,4-bis(4-methoxyphenyl)-1,3,2,4-dithiadiphosphetane-2,4-disulphide as starting materials, the course of the reaction in the process mentioned above can be represented by the following reaction scheme:

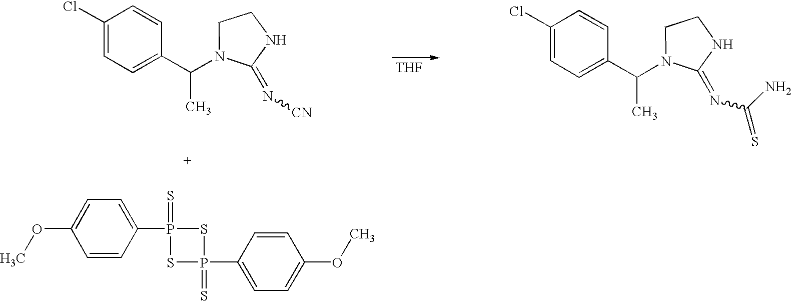

The compounds obtained by processes a), b) and c) can, if appropriate, be alkylated at the heterocycle (see process a)) and/or substituted at the phenyl ring by R¹ (cf., for example, Theil, Angew. Chem. 1999, 111, 2493, Chan et al., Tetrahedron Letters 1998, 39, 2933), using methods known to the person skilled in the art.

It is also possible to oxidize, if appropriate, the compounds obtained by processes a), b) and c) at the sulphur of the heterocycle using methods known to the person skilled in the art to obtain the corresponding sulphinyl and sulphone derivatives (cf., for example, Tanaka et al., Synlett, 1997, 316).

Suitable diluents are inert organic solvents. These include, in particular, aliphatic, alicyclic or aromatic, optionally halogenated hydrocarbons, such as, for example, benzine, benzene, toluene, xylene, anisole, chlorobenzene, dichlorobenzene, petroleum ether, hexane, cyclohexane, dichloromethane, 1,2-dichloroethane, chloroform, carbon tetrachloride, ethers, such as diethyl ether, dioxane, tetrahydrofuran or ethylene glycol dimethyl ether or ethylene glycol diethyl ether, ketones, such as acetone or butanone, nitriles, such as acetonitrile or propionitrile, amnides, such as dimethylformamide, dimethylacetamide, N-methylformanilide, N-methylpyrrolidone or hexamethylphosphoric triamide, esters, such as ethyl acetate, sulphoxides, such as dimethyl sulphoxide or sulpholane, and also alcohols, such as methanol, ethanol or isopropanol. Suitable diluents for carrying out the processes (a), (b) and (c) according to the invention are furthermore water or aqueous solutions. It is also possible to use monophasic or polyphasic mixtures of the abovementioned solvents.

The processes (a), (b) and (c) according to the invention can, if appropriate, be carried out in the presence of a basic reaction auxiliary. Basic reaction auxiliaries are all customary inorganic or organic bases. These include, for example, alkali metal and alkaline earth metal hydroxides, such as sodium hydroxide, potassium hydroxide or calcium hydroxide, alkali metal carbonates or bicarbonates, such as sodium carbonate, potassium carbonate, caesium carbonate or sodium bicarbonate, alkali metal and alkaline earth metal hydrides, such as sodium hydride and calcium hydride, metal alkyls, such as n-, s-, i-butyllithium, and also tertiary amines, such as triethylamine, N,N-dimethylaniline, pyridine, N,N-dimethylaminopyridine, diazabicyclooctane (DABCO), diazabicyclononene (DBN) or diazabicycloundecene (DBU).

When carrying out the processes (a), (b) and (c) according to the invention, the reaction temperatures can be varied within a relatively wide range. In general, the processes are carried out at temperatures between 0° C. and +200° C., preferably at temperatures between +20° C. and +140° C.

When carrying out the processes (a), (b) and (c) according to the invention, the reaction pressure can be varied within a relatively wide range. In general, the processes are carried out at pressures between 0.5 bar and 20 bar, preferably at pressures between atmospheric pressure and 3 bar.

The active compounds, having good plant tolerance and favourable warm-blood toxicity, are suitable for controlling animal pests, in particular insects, arachnids and nematodes, which are encountered in agriculture, in forestry, in the protection of stored products and of materials, and in the hygiene sector. They may be preferably used as crop protection agents. They are active against normally sensitive and resistant species and against all or some stages of development. The abovementioned pests include:

From the order of the Isopoda, for example, Oniscus asellus, Amiadillidium vulgare and Porcellio scaber.

From the order of the Diplopoda, for example, Blaniulus guttulatus.

From the order of the Chilopoda, for example, Geophilus carpophagus and Scutigera spp.

From the order of the Symphyla, for example, Scutigerella immaculata.

From the order of the Thysanura, for example, Lepisma saccharina.

From the order of the Collembola, for example, Onychiurus armatus.

From the order of the Orthoptera, for example, Acheta domesticus, Gryllotalpa spp., Locusta migratoria migratorioides, Melanoplus spp. and Schistocerca gregaria.

From the order of the Blattaria, for example, Blatta orientalis, Periplaneta americana, Leucophaea maderae and Blattella germanica.

From the order of the Dermaptera, for example, Forficula auricularia.

From the order of the Isoptera, for example, Reticulitermes spp.

From the order of the Phthiraptera, for example, Pediculus humanus corporis, Haematopinus spp., Linognathus spp., Trichodectes spp. and Damalinia spp.

From the order of the Thysanoptera, for example, Hercinothrips femoralis, Thrips tabaci, Thrips palmi and Frankliniella accidentalis.

From the order of the Heteroptera, for example, Eurygaster spp., Dysdercus intermedius, Piesma quadrata, Cimex lectularius, Rhodnius prolixus and Triatoma spp.

From the order of the Homoptera, for example, Aleurodes brassicae, Bemisia tabaci, Trialeurodes vaporariorum, Aphis gossypii, Brevicoryne brassicae, Cryptomyzus ribis, Aphis fabae, Aphis pomi, Eriosoma lanigerum, Hyalopterus arundinis, Phylloxera vastatrix, Pemphigus spp., Macrosiphum avenae, Myzus spp., Phorodon humuli, Rhopalosiphum padi, Empoasca spp., Euscelis bilobatus, Nephotettix cincticeps, Lecanium comi, Saissetia oleae, Laodelphax striatellus, Nilaparvata lugens, Aonidiella aurantii, Aspidiotus hederae, Pseudococcus spp. and Psylla spp.

From the order of the Lepidoptera, for example, Pectinophora gossypiella, Bupalus piniarius, Cheimatobia brumata, Lithocolletis blancardella, Hyponomeuta padella, Plutella xylostella, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Mamestra brassicae, Panolis flammea, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofmannophila pseudospretella, Cacoecia podana, Capua reticulana, Choristoneura fuimiferana, Clysia ambiguella, Homona magnanima, Tortrix viridana, Cnaphalocerus spp. and Oulema oryzae.

From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp.,

Psylliodes chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrhynchus assimilis, Hypera postica, Dermestes spp., Trogoderma spp., Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica and Lissorhoptrus oryzophilus.

From the order of the Hymenoptera, for example, Diprion spp., Hoplocampa spp., Lasius spp., Monomorium pharaonis and Vespa spp.

From the order of the Diptera, for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa, Hylemyia spp. and Liriomyza spp.

From the order of the Siphonaptera, for example, Xenopsylla cheopis and Ceratophyllus spp.

From the class of the Arachnida, for example, Scorpio maurus, Latrodectus mactans, Acarus siro, Argas spp., Ornithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Hemitarsonemus spp. and Brevipalpus spp.

The plant-parasitic nematodes include, for example, Pratylenchus spp., Radopholus similis, Ditylenchus dipsaci, Tylenchulus semipenetrans, Heterodera spp., Globodera spp., Meloidogyne spp., Aphelenchoides spp., Longidorus spp., Xiphinema spp., Trichodorus spp. and Bursaphelenchus spp.

The compounds of the formula (I) according to the invention can be used with particularly good results for controlling plant-damaging nematodes, such as, for example, against Meloidogyne incognita larvae, and also for controlling plant-damaging insects, such as, for example, against the peach aphid (Myzus persicae), the larvae of the mustard beetle (Phaedon cochleariae) and also against the caterpillars of the army worm (Spodoptera frugiperda) and for controlling plant-damaging spider mites (Tetranychus urticae).

At appropriate application rates, the compounds according to the invention also have flngicidal properties, such as, in particular, against Pyricularia.

At certain concentrations or application rates, the compounds according to the invention may, if appropriate, also be used as herbicides and microbicides, for example as fungicides, antimycotics and bactericides. If appropriate, they may also be used as intermediates or precursors for the synthesis of further active compounds.

All plants and plant parts can be treated in accordance with the invention. Plants are to be understood as meaning in the present context all plants and plant populations such as desired and undesired wild plants or crop plants (inclusive of naturally occurring crop plants). Crop plants can be plants which can be obtained by conventional plant breeding and optimization methods or by biotechnological and recombinant methods or by combinations of these methods, inclusive of the transgenic plants and inclusive of the plant cultivars protectable or not protectable by plant breeders' rights. Plant parts are to be understood to mean all above-ground and underground parts and organs of plants, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offsets and seeds.

As already mentioned above, it is possible to treat all plants and their parts according to the invention. In a preferred embodiment, wild plant species and plant cultivars, or those obtained by conventional biological breeding, such as crossing or protoplast fuision, and parts thereof, are treated. In a further preferred embodiment, transgenic plants and plant cultivars obtained by genetic engineering, if appropriate in combination with conventional methods (Genetically Modified Organisms), and parts thereof are treated. The term “parts” or “parts of plants” or “plant parts” has been explained above.

Particularly preferably, plants of the plant cultivars which are in each case commercially available or in use are treated according to the invention. Plant cultivars are to be understood as meaning plants having novel properties (“traits”) which can be obtained by conventional breeding, by mutagenesis or by recombinant DNA techniques. This can be varieties, Bio- and genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions to be used according to the invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The preferred transgenic plants or plant cultivars (i.e. those obtained by genetic engineering) which are to be treated according to the invention include all plants which, in the genetic modification, received genetic material which imparted particularly advantageous useful traits to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, oilseed rape and also fruit plants (with the fruits apples, pears, citrus fruits and grape-vines), and particular emphasis is given to maize, soya beans, potatoes, cotton and oilseed rape. Traits that are emphasized are in particular increased defence of the plants against insects by toxins formed in the plants, in particular those formed by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (hereinbelow referred to as “Bt plants”). Traits that are also particularly emphasized are the increased defence of the plants to fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucotnm (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned include the varieties sold under the name Clearfield® (for example maize). Of course, these statements also apply to plant cultivars having these or still to be developed genetic traits, which plants will be developed and/or marketed in the future.

The plants listed can be treated according to the invention in a particularly advantageous manner with the compounds of the general formula I or the active compound mixtures according to the invention. The preferred ranges stated above for the active compounds or mixtures also apply to the treatment of these plants. Particular emphasis is given to the treatment of plants with the compounds or the mixtures specifically mentioned in the present text.

Treatment according to the invention of the plants and plant parts with the active compounds is carried out directly or by allowing the compounds to act on their surroundings, environment or storage space by the customary treatment methods, for example by immersion, spraying, evaporation, fogging, scattering, painting on and, in the case of propagation material, in particular in the case of seed, also by applying one or more coats.

The active compounds can be converted into the customary formulations, such as solutions, emulsions, wettable powders, suspensions, powders, dusts, pastes, soluble powders, granules, suspension-emulsion concentrates, natural and synthetic materials impregnated with active compound, and microencapsulations in polymeric substances.

These formulations are produced in a known manner, for example by mixing the active compounds with extenders, that is liquid solvents and/or solid carriers, optionally with the use of surfactants, that is emulsifiers and/or dispersants, and/or foam-formers.

If the extender used is water, it is also possible to employ for example organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethylformamide and dimethyl sulphoxide, and also water.

Suitable solid carriers are:

for example ammonium salts and ground natural minerals such as kaolins, clays, talc, chalk, quartz, attapulgite, montmorillonite or diatomaceous earth, and ground synthetic minerals, such as highly disperse silica, alumina and silicates; suitable solid carriers for granules are: for example crushed and fractionated natural rocks such as calcite, marble, pumice, sepiolite and dolomite, and also synthetic granules of inorganic and organic meals, and granules of organic material such as sawdust, coconut shells, maize cobs and tobacco stalks; suitable emulsifiers and/or foam-formers are: for example nonionic and anionic emulsifiers, such as polyoxyethylene fatty acid esters, polyoxy-ethylene fatty alcohol ethers, for example alkylaryl polyglycol ethers, alkylsulphonates, alkyl sulphates, arylsulphonates and also protein hydrolysates; suitable dispersants are: for example lignin-sulphite waste liquors and methylcellulose.

Tackifiers such as carboxymethylcellulose and natural and synthetic polymers in the form of powders, granules or latices, such as gum arabic, polyvinyl alcohol and polyvinyl acetate, as well as natural phospholipids such as cephalins and lecithins, and synthetic phospholipids, can be used in the formulations. Other additives can be mineral and vegetable oils.

It is possible to use colorants such as inorganic pigments, for example iron oxide, titanium oxide and Prussian Blue, and organic dyes, such as alizarin dyes, azo dyes and metal phthalocyanine dyes, and trace nutrients such as salts of iron, manganese, boron, copper, cobalt, molybdenum and zinc.

The formulations generally comprise between 0.1 and 95% by weight of active compound, preferably between 0.5 and 90%.

The active compounds according to the invention can be used as such or in their formulations as a mixture with known fungicides, bactericides, acaricides, nematicides, or insecticides, to widen the activity spectrum or to prevent development of resistance, for example. In many cases, synergistic effects are obtained thereby, i.e. the activity of the mixture is greater than the activity of the individual components.

Suitable examples of co-components in mixtures are the following compounds:

Fungicides:

aldimorph, ampropylfos, ampropylfos-potassium, andoprim, anilazine, azaconazole, azoxystrobin,

benalaxyl, benodanil, benomyl, benzamacril, benzamacril-isobutyl, bialaphos, binapacryl, biphenyl, bitertanol, blasticidin-S, bromuconazole, bupirimate, buthiobate,

calcium polysulphide, capsimycin, captafol, captan, carbendazim, carboxin, carvon, quinomethionate, chlobenthiazone, chlorfenazole, chloroneb, chloropicrin, chlorothalonil, chlozolinate, clozylacon, cufraneb, cymoxanil, cyproconazole, cyprodinil, cyprofuram,

debacarb, dichlorophen, diclobutrazole, diclofluanid, diclomezine, dicloran, diethofencarb, difenoconazole, dimethirimol, dimethomorph, diniconazole, diniconazole-M, dinocap, diphenylamine, dipyrithione, ditalimfos, dithianon, dodemorph, dodine, drazoxolon,

edifenphos, epoxiconazole, etaconazole, ethirimol, etridiazole,

famoxadon, fenapanil, fenarimol, fenbuconazole, fenfuram, fenitropan, fenpiclonil, fenpropidin, fenpropimorph, fentin acetate, fentin hydroxide, ferbam, ferimzone, fluazinam, flumetover, fluoromide, fluquinconazole, flurprimidol, flusilazole, flusulfamide, flutolanil, flutriafol, folpet, fosetyl-aluminium, fosetyl-sodium, fthalide, fuberidazole, furalaxyl, furametpyr, furcarbonil, furconazole, furconazole-cis, furmecyclox,

guazatine,

hexachlorobenzene, hexaconazole, hymexazole,

imazalil, imibenconazole, iminoctadine, irninoctadine aIbesilate, iminoctadine triacetate, iodocarb, ipconazole, iprobenfos (IBP), iprodione, irumamycin, isoprothiolane, isovaledione,

kasugamycin, kresoxim-methyl, copper preparations, such as: copper hydroxide, copper naphthenate, copper oxychloride, copper sulphate, copper oxide, oxine-copper and Bordeaux mixture,

mancopper, mancozeb, maneb, meferimzone, mepanipyrim, mepronil, metalaxyl, metconazole, methasulfocarb, methfuroxam, metiram, metomeclam, metsulfovax, mildiomycin, myclobutanil, myclozolin,

nickel dimethyldithiocarbamate, nitrothal-isopropyl, nuarimol,

ofurace, oxadixyl, oxamocarb, oxolinic acid, oxycarboxim, oxyfenthiin,

paclobutrazole, pefurazoate, penconazole, pencycuron, phosdiphen, picoxystrobin, pimaricin, piperalin, polyoxin, polyoxorim, probenazole, prochloraz, procymidone, propamocarb, propanosine-sodium, propiconazole, propineb, pyraclostrobin, pyrazophos, pyrifenox, pyrimethanil, pyroquilon, pyroxyfur,

quinconazole, quintozene (PCNB),

sulphur and sulphur preparations,

tebuconazole, tecloftalam, tecnazene, tetcyclacis, tetraconazole, thiabendazole, thicyofen, thifluzamide, thiophanate-methyl, thiram, tioxymid, tolclofos-methyl, tolylfluanid, triadimefon, triadimenol, triazbutil, triazoxide, trichlamide, tricyclazole, tridemorph, trifloxystrobin, triflumizole, triforine, triticonazole,

uniconazole,

validamycin A, vinclozolin, viniconazole,

zarilamide, zineb, ziram and also

Dagger G,

OK-8705,

OK-8801,

α-(1,1-dimethylethyl)-β-(2-phenoxyethyl)-1H-1,2,4-triazole-1-ethanol,

α-(2,4-dichlorophenyl)-β-fluoro-β-propyl-1H-1,2,4-triazole-1-ethanol,

α-(2,4-dichlorophenyl)-β-methoxy-α-methyl-1H-1,2,4-triazole-1-ethanol,

α-(5-methyl-1,3-dioxan-5-yl)-β-[[4-(trifluoromethyl)-phenyl]-methylene]-1H-1,2,4-triazole 1-ethanol,

(5RS,6RS)-6-hydroxy-2,2,7,7-tetramethyl-5-(1H-1,2,4-triazol-1-yl)-3-octanone,

(E)-α-(methoxyimino)-N-methyl-2-phenoxy-phenylacetamide,

1-isopropyl {2-methyl-1-[[[1-(4-methylphenyl)-ethyl]-amino]-carbonyl]-propyl}-carbamate,

1-(2,4-dichlorophenyl)-2-(1H-1,2,4-triazol-1-yl)-ethanone-O-(phenylmethyl)-oxime,

1-(2-methyl-1-naphthalenyl)-1H-pyrrole-2,5-dione,

1-(3,5-dichlorophenyl)-3-(2-propenyl)-2,5-pyrrolidinedione,

1-[(diiodomethyl)-sulphonyl]-4-methyl-benzene,

1-[[2-(2,4-dichlorophenyl)-1,3-dioxolan-2-yl]-methyl]-1H-imidazole,

1-[[2-(4-chlorophenyl)-3-phenyloxiranyl]-methyl]-1H-1,2,4-triazole,

1-[1-[2-[(2,4-dichlorophenyl)-methoxy]-phenyl]-ethenyl]-1H-imidazole,

1-methyl-5-nonyl-2-(phenylmethyl)-3-pyrrolidinol,

2′,6′-dibromo-2-methyl-4′-trifluoromethoxy-4′-trifluoro-methyl-1,3-thiazole-5-carboxanilide,

2,2-dichloro-N-[1-(4-chlorophenyl)-ethyl]-1-ethyl-3-methyl-cyclopropanecarboxamide,

2,6-dichloro-5-(methylthio)-4-pyrimidinyl-thiocyanate,

2,6-dichloro-N-(4-trifluoromethylbenzyl)-benzamide,

2,6-dichloro-N-[[4-(trifluoromethyl)-phenyl]-methyl]-benzamide,

2-(2,3,3-triiodo-2-propenyl)-2H-tetrazole,

2-[(1-methylethyl)-sulphonyl]-5-(trichloromethyl)-1,3,4-thiadiazole,

2-[[6-deoxy-4-O-(4-O-methyl-β-D-glycopyranosyl)-α-D-glucopyranosyl]-amino]-4-methoxy-1H-pyrrolo[2,3-d]pyrimidine-5-carbonitrile,

2-aminobutane,

2-bromo-2-(bromomethyl)-pentanedinitrile,

2-chloro-N-(2,3-dihydro-1,1,3-trimethyl-1H-inden-4-yl)-3-pyridinecarboxamide,

2-chloro-N-(2,6-dimethylphenyl)-N-(isothiocyanatomethyl)-acetamide,

2-phenylphenol (OPP),

3,4-dichloro-1-[4-(difluoromethoxy)-phenyl]-1H-pyrrole-2,5-dione,

3,5-dichloro-N-[cyano-[(1-methyl-2-propynyl)-oxy]-methyl]-benzamide,

3-(1,1-dimethylpropyl)-1-oxo-1H-indene-2-carbonitrile,

3-[2-(4-chlorophenyl)-5-ethoxy-3-isoxazolidinyl]-pyridine,

4-chloro-2-cyano-N,N-dimethyl-5-(4-methylphenyl)-1H-imidazole-1-sulphonamide,

4-methyl-tetrazolo[1,5-a]quinazolin-5(4H)-one,

8-(1,1-dimethylethyl)-N-ethyl-N-propyl-1,4-dioxaspiro[4.5]decane-2-methanamine,

8-hydroxyquinoline sulphate,

9H-xanthene-2-[(phenylamino)-carbonyl]-9-carboxylic hydrazide,

bis-(1-methylethyl)-3-methyl-4-[(3-methyIbenzoyl)-oxy]-2,5-thiophenedicarboxylate,

cis-1-(4-chlorophenyl)-2-(1H-1,2,4-triazol-1-yl)-cycloheptanol,

cis-4-[3-[4-(1,1-dimethylpropyl)-phenyl-2-methylpropyl]-2,6-dimethyl-morpholinehydrochloride,

ethyl [(4-chlorophenyl)-azo]-cyanoacetate,

potassium hydrogen carbonate,

methanetetrathiol sodium salt,

methyl 1-(2,3-dihydro-2,2-dimethyl-1H -inden-1-yl)-1H-imidazole-5-carboxylate,

methyl N-(2,6-dimethylphenyl)-N-(5-isoxazolylcarbonyl)-DL-alaninate,

methyl N-(chloroacetyl)-N-(2,6-dimethylphenyl)-DL-alaninate,

N-(2,3-dichloro-4-hydroxyphenyl)-1-methyl-cyclohexanecarboxamide,

N-(2,6-dimethylphenyl)-2-methoxy-N-(tetrahydro-2-oxo-3-furanyl)-acetamide,

N-(2,6-dimethylphenyl)-2-methoxy-N-(tetrahydro-2-oxo-3-thienyl)-acetamide,

N-(2-chloro-4-nitrophenyl)-4-methyl-3-nitro-benzenesulfonamide,

N-(4-cyclohexylphenyl)-1,4,5,6-tetrahydro-2-pyrimidinamine,

N-(4-hexylphenyl)-1,4,5,6-tetrahydro-2-pyrimidinamine,

N-(5-chloro-2-methylphenyl)-2-methoxy-N-(2-oxo-3-oxazolidinyl)-acetamide,

N-(6-methoxy)-3-pyridinyl)-cyclopropanecarboxamide,

N-[2,2,2-trichloro-1-[(chloroacetyl)-amino]-ethyl]-benzamide,

N-[3-chloro-4,5-bis-(2-propinyloxy)-phenyl]-N′-methoxy-methanimidamide,

N-formyl-N-hydroxy-DL-alanine sodium salt,

O,O-diethyl [2-(dipropylamino)-2-oxoethyl]-ethylphosphoramidothioate,

O-methyl S-phenyl phenylpropylphosphoramidothioate,

S-methyl 1,2,3-benzothiadiazole-7-carbothioate,

spiro[2H]-1-benzopyrane-2,1′(3′H)-isobenzofuran]-3′-one,

4-[3,4-dimethoxyphenyl)-3-(4-fluorophenyl)-acryloyl]-morpholine.

Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.

Insecticides/Acaricides/Nematicides:

abamectin, acephate, acetamiprid, acrinathrin, alanycarb, aldicarb, aldoxycarb, alphacypermethrin, alphamethrin, amitraz, avermectin, AZ 60541, azadirachtin, azamethiphos, azinphos A, azinphos M, azocyclotin,

Bacillus popilliae, Bacillus sphaericus, Bacillus subtilis, Bacillus thuringiensis, Baculoviruses, Beauveria bassiana, Beauveria tenella, bendiocarb, benfuracarb, bensultap, benzoximate, betacyfluthrin, bifenazate, bifenthrin, bioethanomethrin, biopermethrin, bistrifluron, BPMC, bromophos A, bufencarb, buprofezin, butathiofos, butocarboxim, butylpyridaben, cadusafos, carbaryl, carbofuran, carbophenothion, carbosulfan, cartap, chloethocarb, chlorethoxyfos, chlorfenapyr, chlorfenvinphos, chlorfluazuron, chlormephos, chlorpyrifos, chlorpyrifos M, chlovaporthrin, chromafenozide, cis-resmethrin, cispermethrin, clocythrin, cloethocarb, clofentezine, clothianidine, cyanophos, cycloprene, cycloprothrin, cyfluthrin, cyhalothrin, cyhexatin, cypermethrin, cyromazine,

deltamethrin, demeton M, demeton S, demeton-S-methyl, diafenthiuron, diazinon, dichlorvos, dicofol, diflubenzuron, dimethoate, dimethylvinphos, diofenolan, disulfoton, docusat-sodium, dofenapyn,

eflusilanate, emamectin, empenthrin, endosulfan, Entomopfthora spp., esfenvalerate, ethiofencarb, ethion, ethoprophos, etofenprox, etoxazole, etrimfos,

fenamiphos, fenazaquin, fenbutatin oxide, fenitrothion, fenothiocarb, fenoxacrim, fenoxycarb, fenpropathrin, fenpyrad, fenpyrithrin, fenpyroximate, fenvalerate, fipronil, fluazinam, fluazuron, flubrocythrinate, flucycloxuron, flucythrinate, flufenoxuron, flumethrin, flutenzine, fluvalinate, fonophos, fosmethilan, fosthiazate, fuibfenprox, furathiocarb,

granulosis viruses,

halofenozide, HCH, heptenophos, hexaflumuron, hexythiazox, hydroprene,

imidacloprid, indoxacarb, isazofos, isofenphos, isoxathion, ivermectin,

nuclear polyhedrosis viruses,

lambda-cyhalothrin, lufenuron,

malathion, mecarbam, metaldehyde, methamidophos, metharhizium anisopliae, metharhizium flavoviride, methidathion, methiocarb, methoprene, methomyl, methoxyfenozide, metolcarb, metoxadiazone, mevinphos, miIbemectin, miIbemycin, monocrotophos,

naled, nitenpyram, nithiazine, novaluron,

omethoate, oxamyl, oxydemethon M,

Paecilomyces fumosoroseus, parathion A, parathion M, permethrin, phenthoate, phorate, phosalone, phosmet, phosphamidon, phoxim, pirimicarb, pirimiphos A, pirimiphos M, profenofos, promecarb, propargite, propoxur, prothiofos, prothoate, pymetrozine, pyraclofos, pyresmethrin, pyrethrum, pyridaben, pyridathion, pyrimidifen, pyriproxyfen,

quinalphos,

ribavirin,

salithion, sebufos, silafluofen, spinosad, spirodiclofen, sulfotep, sulprofos,

tau-fluvalinate, tebufenozide, tebufenpyrad, tebupirimiphos, teflubenzuron, tefluthrin, temephos, temivinphos, terbufos, tetrachlorvinphos, tetradifon, thetacypermethrin, thiacloprid, thiamethoxam, thiapronil, thiatriphos, thiocyclam hydrogen oxalate, thiodicarb, thiofanox, thuringiensin, tralocythrin, tralomethrin, triarathene, triazamate, triazophos, triazurone, trichlophenidine, trichlorfon, triflumuron, trimethacarb,

vamidothion, vaniliprole, Verticillium lecanii,

YI 5302,

zeta-cypermethrin, zolaprofos,

(1R-cis)-[5-(phenyhnethyl)-3-furanyl]-methyl-3-[(dihydro-2-oxo-3(2H)-furanylidene)-methyl]-2,2-dimethylcyclopropanecarboxylate,

(3-phenoxyphenyl)-methyl-2,2,3,3-tetramethylcyclopropanecarboxylate,

1-[(2-chloro-5-thiazolyl)methyl]tetrahydro-3,5-dimethyl-N-nitro-1,3,5-triazine-2(1H)-imine,

2-(2-chloro-6-fluorophenyl)-4-[4-(1,1-dimethylethyl)phenyl]-4,5-dihydro-oxazole,

2-(acetyloxy)-3-dodecyl-1,4-naphthalenedione,

2-chloro-N-[[[4-(1-phenylethoxy)-phenyl]-amino]-carbonyl]-benzamide,

2-chloro-N-[[[4-(2,2-dichloro-1,1-difluoroethoxy)-phenyl]-amino]-carbonyl]-benzamide,

3-methylphenyl propylcarbamate,

4-[4-(4-ethoxyphenyl)-4-methylpentyl]-1-fluoro-2-phenoxy-benzene,

4-chloro-2-(1,1-dimethylethyl)-5-[[2-(2,6-dimethyl-4-phenoxyphenoxy)ethyl]thio]-3(2H)-pyridazinone,

4-chloro-2-(2-chloro-2-methylpropyl)-5-[(6-iodo-3-pyridinyl)methoxy]-3(2H)-pyridazinone,

4-chloro-5-[(6-chloro-3-pyridinyl)methoxy]-2-(3,4-dichlorophenyl)-3(2H)-pyridazinone,

Bacillus thuringiensis strain EG-2348,

[2-benzoyl-1-(1,1-dimethylethyl)-hydrazinobenzoic acid,

2,2-dimethyl-3-(2,4-dichlorophenyl)-2-oxo-1-oxaspiro[4.5]dec-3-en-4-yl butanoate,

[3-[(6-chloro-3-pyridinyl)methyl]-2-thiazolidinylidene]-cyanamide,

dihydro-2-(nitromethylene)-2H-1,3-thiazine-3(4H)-carboxaldehyde,

ethyl [2-[[1,6-dihydro-6-oxo-1-(phenylmethyl)-4-pyridazinyl]oxy]ethyl]-carbamate,

N-(3,4,4-trifluoro-1-oxo-3-butenyl)-glycine,

N-(4-chlorophenyl)-3-[4-(difluoromethoxy)phenyl]-4,5-dihydro-4-phenyl-1H-pyrazole-1-carboxamide,

N-[(2-chloro-5-thiazolyl)methyl]-N′-methyl-N″-nitro-guanidine,

N-methyl-N′-(1-methyl-2-propenyl)-1,2-hydrazinedicarbothioamide,

N-methyl-N′-2-propenyl-1,2-hydrazinedicarbothioamide,

O,O-diethyl [2-(dipropylamino)-2-oxoethyl]-ethylphosphoramidothioate

3,5-dichloro-1-(3,3-dichloro-2-propenyloxy)-4-[3-(5-trifluoromethylpyridin-2-yloxy)-propoxy]-benzene.

A mixture with other known active compounds, such as herbicides, or with fertilizers and growth regulators is also possible.

The active compounds according to the invention can furthermore be present when used as insecticides in their commercially available formulations and in the use forms, prepared from these formulations, as a mixture with synergistic agents. Synergistic agents are compounds which increase the action of the active compounds, without it being necessary for the synergistic agent added to be active itself.

The active compound content of the use forms prepared from the commercially available formulations can vary within wide limits. The active compound concentration of the use forms can be from 0.0000001 to 95% by weight of active compound, preferably between 0.0001 and 1% by weight.

The compounds are employed in a customary manner appropriate for the use forms.

When used against hygiene pests and pests of stored products, the active compound is distinguished by an excellent residual action on wood and clay as well as a good stability to alkali on limed substrates.

The active compounds according to the invention act not only against plant, hygiene and stored product pests, but also in the veterinary medicine sector against animal parasites (ectoparasites), such as hard ticks, soft ticks, mange mites, leaf mites, flies (biting and licking), parasitic fly larvae, lice, hair lice, feather lice and fleas. These parasites include:

From the order of the Anoplurida, for example, Haematopinus spp., Linognathus spp., Pediculus spp., Phtirus spp. and Solenopotes spp.

From the order of the Mallophagida and the suborders Amblycerina and Ischnocerina, for example, Trimenopon spp., Menopon spp., Trinoton spp., Bovicola spp., Werneckiella spp., Lepikentron spp., Damalina spp., Trichodectes spp. and Felicola spp.

From the order Diptera and the suborders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.

From the order of the Siphonapterida, for example, Pulex spp., Ctenocephalides spp., Xenopsylla spp. and Ceratophyllus spp.

From the order of the Heteropterida, for example, Cimex spp., Triatoma spp., Rhodnius spp. and Panstrongylus spp.

From the order of the Blattarida, for example, Blatta orientalis, Periplaneta americana, Blattela germanica and Supella spp.

From the subclass of the Acaria (Acarida) and the orders of the Meta- and Mesostigmata, for example, Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp, Raillietia spp, Pneumonyssus spp, Stemostoma spp and Varroa spp

From the order of the Actinedida (Prostigmata) and Acaridida (Astigmata), for example, Acarapis spp, Cheyletiella spp, Ornithocheyletia spp, Myobia spp, Psorergates spp, Demodex spp, Trombicula spp, Listrophorus spp, Acarus spp, Tyrophagus spp, Caloglyphus spp, Hypodectes spp, Pterolichus spp, Psoroptes spp, Chorioptes spp, Otodectes spp, Sarcoptes spp, Notoedres spp, Knemidocoptes spp, Cytodites spp and Laminosioptes spp.

The active compounds of the formula (I) according to the invention are also suitable for controlling arthropods which infest agricultural productive livestock, such as, for example, cattle, sheep, goats, horses, pigs, donkeys, camels, buffalo, rabbits, chickens, turkeys, ducks, geese and bees, other pets, such as, for example, dogs, cats, caged birds and aquarium fish, and also so-called test animals, such as, for example, hamsters, guinea pigs, rats and mice. By controlling these arthropods, cases of death and reduction in productivity (for meat, milk, wool, hides, eggs, honey etc.) should be diminished, so that more economic and easier animal husbandry is possible by use of the active compounds according to the invention.

The active compounds according to the invention are used in the veterinary sector in a known manner by enteral administration in the form of, for example, tablets, capsules, potions, drenches, granules, pastes, boluses, the feed-through process and suppositories, by parenteral administration, such as, for example, by injection (intramuscular, subcutaneous, intravenous, intraperitoneal and the like), implants, by nasal administration, by dermal use in the form, for example, of dipping or bathing, spraying, pouring on and spotting on, washing and powdering, and also with the aid of moulded articles containing the active compound, such as collars, ear marks, tail marks, limb bands, halters, marking devices and the like.

When used for cattle, poultry, pets and the like, the active compounds of the formula (I) can be used as formulations (for example powders, emulsions, free-flowing compositions), which comprise the active compounds in an amount of 1 to 80% by weight, directly or after 100 to 10 000-fold dilution, or they can be used as a chemical bath.

It has furthermore been found that the compounds according to the invention also have a strong insecticidal action against insects which destroy industrial materials.

The following insects may be mentioned as examples and as being preferred—but without any limitation:

Beetles, such as

Hylotrupes bajulus, Chlorophorus pilosis, Anobium punctatum, Xestobium rufovillosum, Ptilinus pecticornis, Dendrobium pertinex, Emobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec., Tryptodendron spec., Apate monachus, Bostrychus capucins, Heterobostrychus brunneus, Sinoxylon spec. and Dinoderus minutus.

Hymenopterons, such as

Sirex juvencus, Urocerus gigas, Urocerus gigas taignus and Urocerus augur.

Termites, such as

Kalotennes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis and Coptoterrnes formosanus.

Bristletails, such as Lepisma saccharina.

Industrial materials in the present context are to be understood as meaning non-living materials, such as, preferably, plastics, adhesives, sizes, papers and cards, leather, wood and processed wood products and coating compositions.

Wood and processed wood products are materials to be protected, especially preferably, from insect infestation.

Wood and processed wood products which can be protected by the agent according to the invention or mixtures comprising this are to be understood as meaning, for example:

building timber, wooden beams, railway sleepers, bridge components, boat jetties, wooden vehicles, boxes, pallets, containers, telegraph poles, wood panelling, wooden windows and doors, plywood, chipboard, joinery or wooden products which are used quite generally in house-building or in building joinery.

The active compounds can be used as such, in the form of concentrates or in generally customary formulations, such as powders, granules, solutions, suspensions, emulsions or pastes.

The formulations mentioned can be prepared in a manner known per se, for example by mixing the active compounds with at least one solvent or diluent, emulsifier, dispersing agent and/or binder or fixing agent, a water repellent, if appropriate siccatives and UV stabilizers and if appropriate dyes and pigments, and also other processing auxiliaries.

The insecticidal compositions or concentrates used for the preservation of wood and wood-derived timber products comprise the active compound according to the invention in a concentration of 0.0001 to 95% by weight, in particular 0.001 to 60% by weight.

The amount of the compositions or concentrates employed depends on the nature and occurrence of the insects and on the medium. The optimum amount employed can be determined for the use in each case by a series of tests. In general, however, it is sufficient to employ 0.0001 to 20% by weight, preferably 0.001 to 10% by weight, of the active compound, based on the material to be preserved.

Solvents and/or diluents which are used are an organic chemical solvent or solvent mixture and/or an oily or oil-like organic chemical solvent or solvent mixture of low volatility and/or a polar organic chemical solvent or solvent mixture and/or water, and if appropriate an emulsifier and/or wetting agent.

Organic chemical solvents which are preferably used are oily or oil-like solvents having an evaporation number above 35 and a flashpoint above 30° C, preferably above 45° C. Substances which are used as such oily or oil-like water-insoluble solvents of low volatility are appropriate mineral oils or aromatic fractions thereof, or solvent mixtures containing mineral oils, preferably white spirit, petroleum and/or alkyIbenzene.

Mineral oils having a boiling range from 170 to 220° C., white spirit having a boiling range from 170 to 220° C., spindle oil having a boiling range from 250 to 350° C., petroleum and aromatics having a boiling range from 160 to 280° C., turpentine oil and the like, are advantageously employed.

In a preferred embodiment, liquid aliphatic hydrocarbons having a boiling range from 180 to 210° C. or high-boiling mixtures of aromatic and aliphatic hydrocarbons having a boiling range from 180 to 220° C. and/or spindle oil and/or monochloronaphthalene, preferably α-monochloronaphthalene, are used.

The organic oily or oil-like solvents of low volatility which have an evaporation number above 35 and a flashpoint above 30° C., preferably above 45° C., can be replaced in part by organic chemical solvents of high or medium volatility, providing that the solvent mixture likewise has an evaporation number above 35 and a flashpoint above 30° C., preferably above 45° C., and that the insecticide/flngicide mixture is soluble or emulsifiable in this solvent mixture.

According to a preferred embodiment, some of the organic chemical solvent or solvent mixture is replaced by an aliphatic polar organic chemical solvent or solvent mixture. Aliphatic organic chemical solvents containing hydroxyl and/or ester and/or ether groups, such as, for example, glycol ethers, esters or the like, are preferably used.

Organic chemical binders which are used in the context of the present invention are the synthetic resins and/or binding drying oils which are known per se, are water-dilutable and/or are soluble or dispersible or emulsifiable in the organic chemical solvents employed, in particular binders consisting of or comprising an acrylate resin, a vinyl resin, for example polyvinyl acetate, polyester resin, polycondensation or polyaddition resin, polyurethane resin, alkyd resin or modified alkyd resin, phenolic resin, hydrocarbon resin, such as indene-cumarone resin, silicone resin, drying vegetable oils and/or drying oils and/or physically drying binders based on a natural and/or synthetic resin.

The synthetic resin used as the binder can be employed in the form of an emulsion, dispersion or solution. Bitumen or bituminous substances can also be used as binders in an amount of up to 10% by weight. Dyestuffs, pigments, water-repelling agents, odour correctants and inhibitors or anticorrosive agents and the like which are known per se can additionally be employed.

It is preferred according to the invention for the composition or concentrate to comprise, as the organic chemical binder, at least one alkyd resin or modified alkyd resin and/or one drying vegetable oil. Alkyd resins having an oil content of more than 45% by weight, preferably 50 to 68% by weight, are preferably used according to the invention.

All or some of the binder mentioned can be replaced by a fixing agent (mixture) or a plasticizer (mixture). These additives are intended to prevent evaporation of the active compounds and crystallization or precipitation. They preferably replace 0.01 to 30% of the binder (based on 100% of the binder employed).

The plasticizers originate from the chemical classes of phthalic acid esters, such as dibutyl, dioctyl or benzyl butyl phthalate, phosphoric acid esters, such as tributyl phosphate, adipic acid esters, such as di-(2-ethylhexyl) adipate, stearates, such as butyl stearate or amyl stearate, oleates, such as butyl oleate, glycerol ethers or higher molecular weight glycol ethers, glycerol esters and p-toluenesulphonic acid esters.

Fixing agents are based chemically on polyvinyl alkyl ethers, such as, for example, polyvinyl methyl ether or ketones, such as benzophenone or ethylenebenzophenone.

Possible solvents or diluents are, in particular, also water, if appropriate as a mixture with one or more of the abovementioned organic chemical solvents or diluents, emulsifiers and dispersing agents.

Particularly effective preservation of wood is achieved by impregnation processes on a large industrial scale, for example vacuum, double vacuum or pressure processes.

The ready-to-use compositions can also comprise other insecticides, if appropriate, and also one or more fingicides, if appropriate.

Possible additional mixing partners are, preferably, the insecticides and fungicides mentioned in WO 94/29 268. The compounds mentioned in this document are an explicit constituent of the present application.

Especially preferred mixing partners which may be mentioned are insecticides, such as chlorpyriphos, phoxim, silafluofin, alphamethrin, cyfluthrin, cypermethrin, deltamethrin, permethrin, imidacloprid, NI-25, flufenoxuron, hexaflumuron, transfluthrin, thiacloprid, methoxyphenoxide and triflumuron,

and also fungicides, such as epoxyconazole, hexaconazole, azaconazole, propiconazole, tebuconazole, cyproconazole, metconazole, imazalil, dichlorfluanid, tolylfluanid, 3-iodo-2-propinyl-butyl carbamate, N-octyl-isothiazolin-3-one and 4,5-di-chloro-N-octylisothiazolin-3-one.

The compounds according to the invention can at the same time be employed for protecting objects which come into contact with salt water or brackish water, such as hulls, screens, nets, buildings, moorings and signalling systems, against fouling.

Fouling by sessile Oligochaeta, such as Serpulidae, and by shells and species from the Ledamorpha group (goose barnacles), such as various Lepas and Scalpellum species, or by species from the Balanomorpha group (acorn barnacles), such as Balanus or Pollicipes species, increases the frictional drag of ships and, as a consequence, leads to a marked increase in operation costs owing to higher energy consumption and additionally frequent residence in the dry dock.

Apart from fouling by algae, for example Ectocarpus sp. and Ceramium sp., fouling by sessile Entomostraka groups, which come under the generic term Cirripedia (cirriped crustaceans), is of particular importance.

Surprisingly, it has now been found that the compounds according to the invention, alone or in combination with other active compounds, have an outstanding antifouling action.

Using the compounds according to the invention, alone or in combination with other active compounds, allows the use of heavy metals such as, for example, in bis-(trialkyltin) sulphides, tri-n-butyltin laurate, tri-n-butyltin chloride, copper(I) oxide, triethyltin chloride, tri-n-butyl(2-phenyl-4-chlorophenoxy)tin, tributyltin oxide, molybdenum disulphide, antimony oxide, polymeric butyl titanate, phenyl-(bispyridine)bismuth chloride, tri-n-butyltin fluoride, manganese ethylenebisthiocarbamate, zinc dimethyldithiocarbamate, zinc ethylenebisthiocarbamate, zinc salts and copper salts of 2-pyridinethiol 1-oxide, bisdimethyldithiocarbamoylzinc ethylene-bisthiocarbamate, zinc oxide, copper(I) ethylene-bisdithiocarbamate, copper thiocyanate, copper naphthenate and tributyltin halides to be dispensed with, or the concentration of these compounds substantially reduced.

If appropriate, the ready-to-use antifouling paints can additionally comprise other active compounds, preferably algicides, fingicides, herbicides, molluscicides, or other antifouling active compounds.

Preferably suitable components in combinations with the antifouling compositions according to the invention are:

algicides such as

2-tert-butylamino-4-cyclopropylamino-6-methylthio-1,3,5-triazine, dichlorophen, diuron, endothal, fentin acetate, isoproturon, methabenzthiazuron, oxyfluorfen, quinoclamine and terbutryn;

fungicides such as benzo[b]thiophenecarboxylic acid cyclohexylamide S,S-dioxide, dichlofluanid, fluorfolpet, 3-iodo-2-propinyl butylcarbamate, tolylfluanid and azoles such as

azaconazole, cyproconazole, epoxyconazole, hexaconazole, metconazole, propiconazole and tebuconazole;

molluscicides such as

fentin acetate, metaldehyde, methiocarb, niclosamid, thiodicarb and trimethacarb;

or conventional antifouling active compounds such as

4,5-dichloro-2-octyl-4-isothiazolin-3-one, diiodomethylparatryl sulphone, 2-(N,N-di-methylthiocarbamoylthio)-5-nitrothiazyl, potassium, copper, sodium and zinc salts of 2-pyridinethiol 1-oxide, pyridine-triphenyIborane, tetrabutyldistannoxane, 2,3,5,6-tetrachloro-4-(methylsulphonyl)-pyridine, 2,4,5,6-tetrachloroisophthalonitrile, tetramethylthiuram disulphide and 2,4,6-trichlorophenylmaleimide.

The antifouling compositions used comprise the active compound according to the invention of the compounds according to the invention in a concentration of 0.001 to 50% by weight, in particular 0.01 to 20% by weight.

Moreover, the antifouling compositions according to the invention comprise the customary components such as, for example, those described in Ungerer, Chem. Ind. 1985, 37, 730-732 and Williams, Antifouling Marine Coatings, Noyes, Park Ridge, 1973.

Besides the algicidal, fungicidal, molluscicidal active compounds and insecticidal active compounds according to the invention, antifouling paints comprise, in particular, binders.

Examples of recognized binders are polyvinyl chloride in a solvent system, chlorinated rubber in a solvent system, acrylic resins in a solvent system, in particular in an aqueous system, vinyl chloride/vinyl acetate copolymer systems in the form of aqueous dispersions or in the form of organic solvent systems, butadiene/styrene/acrylonitrile rubbers, drying oils such as linseed oil, resin esters or modified hardened resins in combination with tar or bitumens, asphalt and epoxy compounds, small amounts of chlorine rubber, chlorinated polypropylene and vinyl resins.

If appropriate, paints also comprise inorganic pigments, organic pigments or colorants which are preferably insoluble in salt water. Paints may furthermore comprise materials such as colophonium to allow controlled release of the active compounds. Furthermore, the paints may comprise plasticizers, modifiers which affect the rheological properties and other conventional constituents. The compounds according to the invention or the abovementioned mixtures may also be incorporated into self-polishing antifouling systems.

The active compounds are also suitable for controlling animal pests, in particular insects, arachnids and mites, which are found in enclosed spaces such as, for example, dwellings, factory halls, offices, vehicle cabins and the like. They can be employed in domestic insecticide products for controlling these pests alone or in combination with other active compounds and auxiliaries. They are active against sensitive and resistant species and against all developmental stages. These pests include:

From the order of the Scorpionidea, for example, Buthus occitanus.

From the order of the Acarina, for example, Argas persicus, Argas reflexus, Bryobia ssp., Dermanyssus gallinae, Glyciphagus domesticus, Ornithodorus moubat, Rhipicephalus sanguineus, Trombicula alfreddugesi, Neutrombicula autumnalis, Dermatophagoides pteronissimus, Dermatophagoides forinae.

From the order of the Araneae, for example, Aviculariidae, Araneidae.

From the order of the Opiliones, for example, Pseudoscorpiones chelifer, Pseudoscorpiones cheiridium, Opiliones phalangium.

From the order of the Isopoda, for example, Oniscus asellus, Porcellio scaber.

From the order of the Diplopoda, for example, Blaniulus guttulatus, Polydesmus spp.

From the order of the Chilopoda, for example, Geophilus spp.

From the order of the Zygentoma, for example, Ctenolepisma spp., Lepisma saccharina, Lepismodes inquilinus.

From the order of the Blattaria, for example, Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa.

From the order of the Saltatoria, for example, Acheta domesticus.

From the order of the Dermaptera, for example, Forficula auricularia.

From the order of the Isoptera, for example, Kalotermes spp., Reticulitermes spp.

From the order of the Psocoptera, for example, Lepinatus spp., Liposcelis spp.

From the order of the Coleptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais, Stegobium paniceum.

From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga camaria, Simulium spp., Stomoxys calcitrans, Tipula paludosa.

From the order of the Lepidoptera, for example, Achroia grisella, Galleria mellonella, Plodia interpunctella, Tinea cloacella, Tinea pellionella, Tineola bisselliella.

From the order of the Siphonaptera, for example, Ctenocephalides canis, Ctenocephalides felis, Pulex irritans, Tunga penetrans, Xenopsylla cheopis.

From the order of the Hymenoptera, for example, Camponotus herculeanus, Lasius fuliginosus, Lasius niger, Lasius umbratus, Monomorium pharaonis, Paravespula spp., Tetramorium caespitum.

From the order of the Anoplura, for example, Pediculus humanus capitis, Pediculus humanus corporis, Phthirus pubis.

From the order of the Heteroptera, for example, Cimex hemipterus, Cimex lectularius, Rhodinus prolixus, Triatoma infestans.

They are used in the household insecticides sector alone or in combination with other suitable active compounds such as phosphoric esters, carbamates, pyrethroids, growth regulators or active compounds from other known classes of insecticides.

They are used in aerosols, pressure-free spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free, or passive, evaporation systems, moth papers, moth bags and moth gels, as granules or dusts, in baits for spreading or in bait stations.

### PREPARATION EXAMPLES

### Example 1

### 1-[1-(4-Chlorophenyl)ethyl]-2-imidazolidinylidenecyanamide

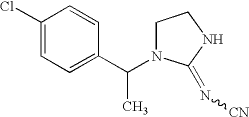

At room temperature, 1.0 g of sodium hydride (60% in paraffin oil) was added a little at a time to a solution of 2.7 g of 2-imidazolidinylidenecyanamide in 50 ml of dimethylformamide, and the mixture was stirred at this temperature for 1 h, and 5.5 g of 1-chloro-4-(1-chloroethyl)benzene were then added. After 8 h at 80° C., the mixture was cooled to room temperature, the solvent was removed under reduced pressure and the residue was taken up in dichloromethane. The resulting solution was washed with water, dried over sodium sulphate and concentrated. Chromatographic purification of the residue gave 1.0 g of 1-[1-(4-chlorophenyl)ethyl]-2-imidazolidinylidenecyanamide of melting point 133° C.

### Example 2

### N-{1-[1-(4-Chlorophenyl)ethyl]-2-imidazolidinylidene}thiourea

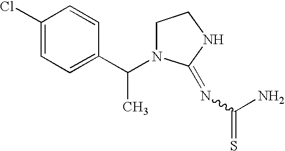

0.6 g of 1-[1-(4-chlorophenyl)ethyl]-2-imidazolidinylidenecyanamide and 1.0 g of 2,4-bis-(4-methoxyphenyl)-1,3,2,4-dithiadiphosphetane-2,4-disulphide in 20 ml of tetrahydrofliran were heated at 50° C. for 4 h. After cooling, the solvent was removed under reduced pressure and the residue was purified chromatographically. This gave 0.4 g of N-{1-[1-(4-chlorophenyl)ethyl]-2-imidazolidinylidene}thiourea of melting point 107° C.

The compounds of the formula (I) listed in Table 1 are obtained analogously to Examples 1 and 2 above and/or in accordance with the general statements concerning the preparation.

TABLE 1
Compounds of the formula (I)

(I)

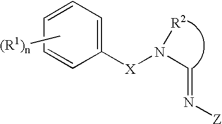

| | | Melting point |
|---|---|---|
| Ex. No. | Structure | or log p |
| 3 | 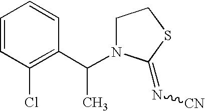 | 86° C. |
| 4 | 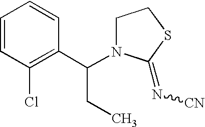 | 139° C. |
| 5 | 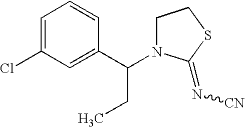 | 85° C. |
| 6 | 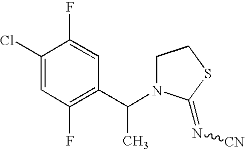 | 138° C. |
| 7 | 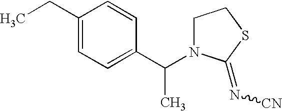 | oil 2.09 |
| 8 | 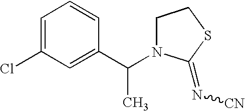 | 84° C. |
| 9 | 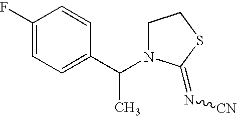 | 64° C. |
| 10 | 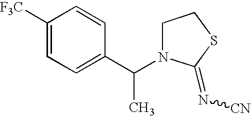 | 131° C. |
| 11 | 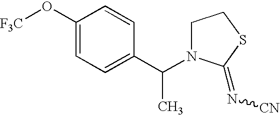 | 78° C. |
| 12 | 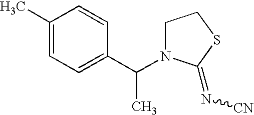 | oil 3.06 |
| 13 | 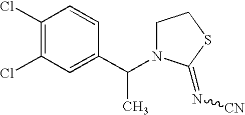 | oil 2.84 |
| 14 | 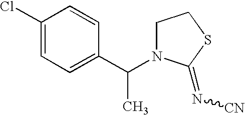 | 66° C. |
| 15 | 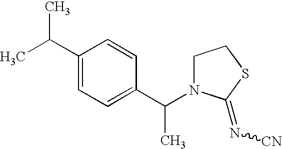 | oil 3.11 |
| 16 | 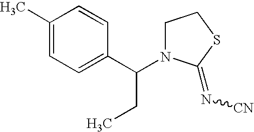 | oil |
| 17 | 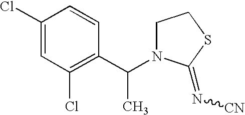 | 134° C. |
| 18 | 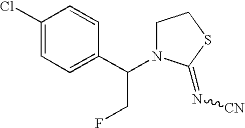 | oil 2.41 |
| 19 | 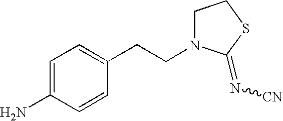 | 123° C. |
| 20 | 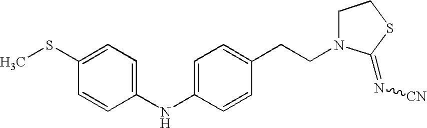 | 135° C. |
| 21 | 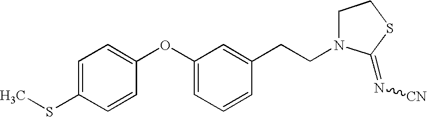 | oil 3.29 |
| 22 | 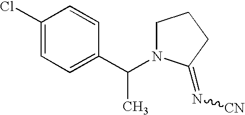 | 82-85° C. |
| 23 | 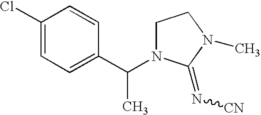 | oil 2.29 |
| 24 | 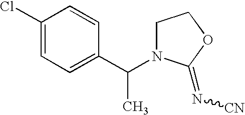 | 111° C. |
| 25 | 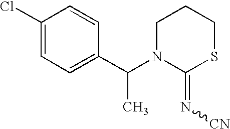 | 128° C. |
| 26 | 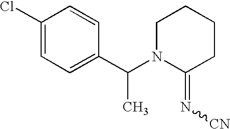 | 115-117° C. |
| 27 | 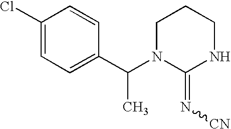 | 180° C. |
| 28 | 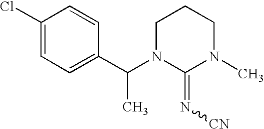 | oil 2.23 |
| 29 | 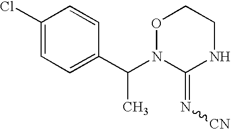 | 171° C. |
| 30 | 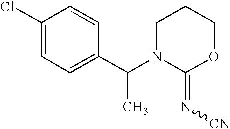 | 103° C. |
| 31 | 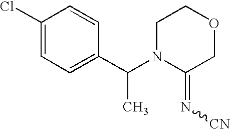 | 112° C. |
| 32 | 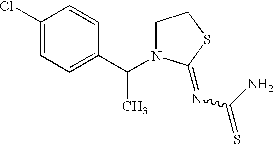 | 149° C. |
| 33 | 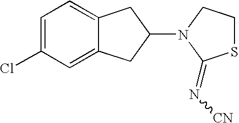 | 2.68 |
| 34 | 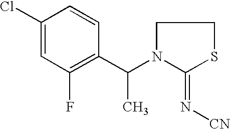 | 124-125° C. |
| 35 | 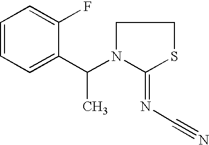 | 100° C. |
| 36 | 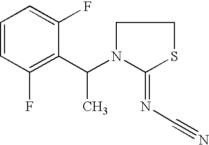 | 130° C. |
| 37 | 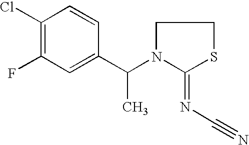 | 87° C. |
| 38 | 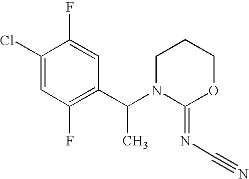 | 112° C. |
| 39 | 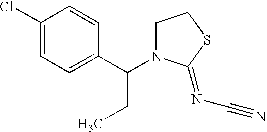 | 2.81 |
| 40 | 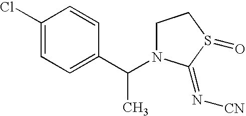 | 1.86 |
| 41 | 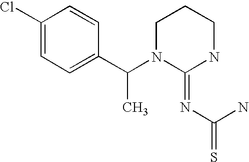 | 1.48 |
| 42 | 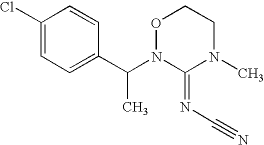 | 2.21 |
| 43 | 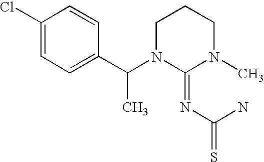 | 1.48 |
| 44 | 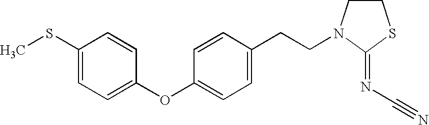 | 49° C. |
| 45 | 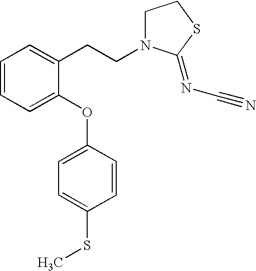 | 3.33 |
| 46 | 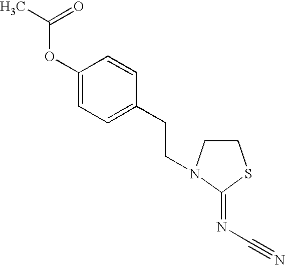 | 1.89 |
| 47 | 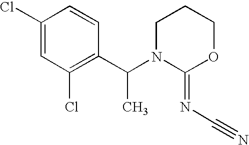 | 2.43 |
| 48 | 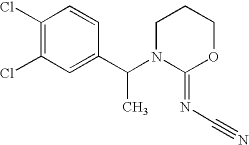 | 2.59 |
| 49 | 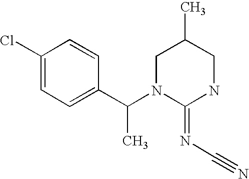 | 2.65 |
| 50 | 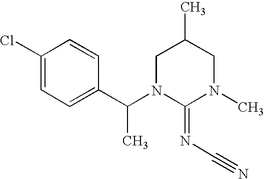 | 2.52 |
| 51 | 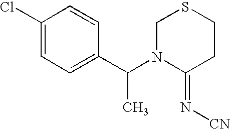 | 2.63 |
| 52 | 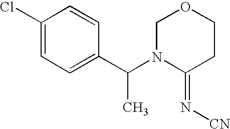 | 2.28 |

The log P values given in Table 1 were determined in accordance with EEC Directive 79/831 Annex V.A8 by HPLC (High Performance Liquid Chromatography) using a reversed-phase column (C18).

Temperature: 43° C.

Mobile phase: 0.1% aqueous phosphoric acid, acetonitrile; linear gradient from 10% acetonitrile to 90% acetonitrile

Calibration was carried out using unbranched alkan-2-ones (having 3 to 16 C atoms) with known log P values. (Determination of the log P values by the retention times using linear interpolation between two successive alkanones).

Starting Materials of the Formula (II)

### Example (II-1)

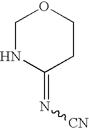

In the presence of 1.9 g of silver oxide (the flask has to be shielded against light), 0.7 g of 1,3-oxazin-4-one and 3.3 ml of methyl iodide were stirred at room temperature for about 5 h. Excess methyl iodide was removed under reduced pressure, and the residue was then taken up in tetrahydrofuran and the mixture was filtered. After concentration of the filtrate, a solution of 0.3 g of cyanamide in 6.5 ml of tetrahydrofuran was added to the residue, and the mixture was heated at reflux for 3 h. After cooling, the mixture was concentrated under reduced pressure and the crude product was purified chromatographically. This gave 15 mg of 1,3-oxazin-4-ylidenecyanamide.

LC-MS mz (%)=126 (MH⁺, 100).

¹H-NMR (600 MHz, CD₃CN, δ)=2.81; 3.95 (4H, 2× —CH₂—); 4.75 (2H, —N—CH₂—O—); 7.64 (br. 1H, —NH—) ppm.

### Example (11-2)

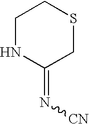

3.0 g of 5-(methylsulphanyl)-3,6-dihydro-2H-1,4-thiazine and 1.0 g of cyanamide in 50 ml of tetrahydrofuran were heated at reflux for 3 h. After cooling, the mixture was concentrated under reduced pressure and the residue was purified chromatographically. This gave 1.5 g of 3-thiomorpholinylidenecyanamide (mixture of tautomers).

EI-MS mz (%)=141 (M⁺, 100).

¹H-NMR (400 MHz, DMSO-d₆, δ)=2.8; 3.4; 3.6 (3× —CH₂—); 9.35 (1H, NH—) ppm (signals of the main tautomer).

### Example (II-3)

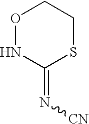

1.0 g of 2-aminooxyethanethiol hydrochloride was added to a mixture of 60 ml of ethanol and 45 ml of water. 0.9 g of potassium carbonate and 0.6 g of methyl N-cyanodithiocarbamate were added, and the mixture was then stirred at room temperature for 4 h. The mixture was concentrated under reduced pressure, and the residue was then stirred with diisopropyl ether and the solid was filtered off with suction. This gave 1.3 g of 1,4,2-oxathiazin-3-ylidenecyanamide (contains salt). This crude product was used without further purification for the subsequent reaction.

LC-MS mz (%)=144 (MH⁺, 100); UV (λₘₐₓ)=255.5 nm.

### Example (11-4)

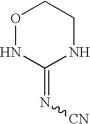

3.0 g of 2-aminoethylhydroxylamine dihydrochloride, 5.6 g of potassium carbonate and 3.3 g of methyl N-cyanodithiocarbamate were added to a mixture of 900 ml of water and 640 ml of ethanol. The mixture was heated at about 40° C. for 3 h. After cooling, the mixture was concentrated under reduced pressure and the residue was used without further purification for subsequent reactions.

LC-MS mz (%)=127 (MH⁺, 100); UV (λₘₐₓ)=226 nm.

### Example (11-5)

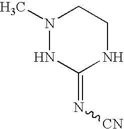

4.0 g of 1-(2-aminoethyl)-1-methylhydrazine were dissolved in 150 ml of ethanol and, at 0° C., 6.5 g of methyl N-cyanodithiocarbamate were added. After 18 h at room temperature, the mixture was cooled to 0° C. and the precipitated product was separated off and dried. This gave 4.9 g of 1-methyl-1,2,4-triazin-3-ylidenecyanamide.

LC-MS mz (%)=MH⁺, 100).

¹H-NMR (400 MHz, CD₃CN, δ)=2.58 (3H, —N—CH₃) 3.00; 3.38 (2× 2H; —N—CH₂—); 6.3; 7.4 (br. 2× 1H, —NH—) ppm.

### Use Examples

### Example A

Meloidogyne Test/Aqueous System

To prepare a suitable preparation of active compound, 1 part by weight of active compound is mixed with 1 000 parts by weight of methanol, and per preparation to be tested, 10 μl are added to a well of a 96-well plate (from Greiner), where the solvent evaporates.

In the 96-well plates, the wells containing the active compound are filled with lecithin solution (0.01%) and Meloidogyne incognita larvae suspension. The concentration of active compound is now 40 ppm.

After the time indicated in the table, the nematicidal action is determined in % by the activity of the larvae. 100% means that no activity has been found; 0% means that the activity in the treated wells corresponds to that of the untreated control.

In this test, for example, the following compound of the Preparation Examples shows good activity:

TABLE 2
Plant-damaging nematodes Meloidogyne test/aqueous system

| Active compound | Concentration of active | Effect in % after |
|---|---|---|
| Ex. No | compound in ppm | 4 days |
| 14 | 40 | 100 |

### Example B

Meloidogyne Test

To prepare a suitable preparation of active compound, 1 part by weight of active compound is mixed with 30 parts by weight of dimethylformamide and 1 part by weight of emulsifier (alllylaryl polyglycol ether), and the concentrate is diluted with water to the desired concentration.

Containers are filled with sand, solution of active compound, Meloidogyne incognita egg/larvae suspension and lettuce seeds. The lettuce seeds germinate and the plants develop. On the roots, galls are formed.

After the period of time indicated in the table, the nematicidal action is determined in % using gall formation as a measure. 100% means that no galls were found; 0% means that the number of galls on the treated plants corresponds to that of the untreated control.

In this test, for example, the following compounds of the Preparation Examples show good activity:

TABLE 3
Plant-damaging nematodes Meloidogyne test

| Active compound | Concentration of active | Effect in % after |
|---|---|---|
| Ex. No | compound in ppm | 4 days |
| 1 | 20 | 98 |
| 3 | 20 | 100 |
| 4 | 20 | 100 |
| 14 | 20 | 100 |
| 22 | 20 | 100 |
| 23 | 20 | 90 |
| 28 | 20 | 95 |

What is claimed is:

1-10 (Cancelled)

11. A compound of formula (I) 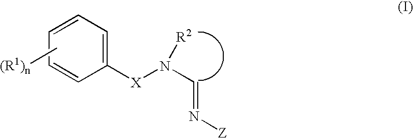
in which
Z represents CN or C(S)NH₂,
X represents substituted methylene or optionally substituted alkylene or alkylidene having in each case 2 to 6 carbon atoms,
where two substituents X together with the C atom or the C atoms to which they are attached may represent cycloalkyl having 3 to 6 carbon atoms, and
where X and R¹ are optionally linked by optionally substituted alkylene or alkylidene having 1 to 3 carbon atoms,
n represents the numbers 1 to 5,
R¹ represents hydroxyl, amino, cyano, nitro, or halogen; represents optionally hydroxyl-, cyano-, or halogen-substituted C₁-C₄-alkyl, C₁-C₄-alkoxy, C₁-C₄-alkylamino, and di-(C₁-C₄)-alkylamino; represents optionally halogen-substituted C₁-C₄-alkyl-carbonyl, C₁-C₄-alkoxy-carbonyl, C₁-C₄-alkyl-carbonyl-amino, C₁-C₄-alkylthio, C₁-C₄-alkylsulphinyl, C₁-C₄-alkylsulphonyl, aminocarbonyl, aminothiocarbonyl, C₁-C₄-alkylaminocarbonyl, di-(C₁-C₄)-alkylamino-carbonyl, aminosulphonyl, C₁-C₄-alkylaminosulphonyl, or di-(C₁-C₄)-alkylamino-sulphonyl; represents phenyl, phenoxy, phenylthio, phenylsulphinyl, phenylsulphonyl, benzyl, phenylamino, pyridyloxy, or pyridylamino, each of which is optionally substituted by hydroxyl, cyano, nitro, halogen, C₁-C₄-alkyl, C₁-C₄-alkoxy, C₁-C₄-alkylthio, C₁-C₄-alkylsulphinyl, C₁-C₄-alkylsulphonyl, C₁-C₄-halogenoalkyl, C₁-C₄-halogenoalkoxy, C₁-C₄-halogenoalkylthio, C₁-C₄-halogenoalkylsulphinyl, or C₁-C₄-halogenoalkylsulphonyl having in each case 1 to 5 identical or different halogen atoms selected from the group consisting of fluorine, chlorine, and bromine,
with the proviso that two radicals R¹ in the 3- and 4-positions of the phenyl ring do not both represent hydroxyl or optionally substituted alkoxy, phenoxy, or pyridyloxy, and
R² together with the N and C atoms to which it is attached represents an optionally C₁-C₆-alkyl-substituted saturated 5- or 6-membered heterocycle having 1 to 3 hetero atoms selected from the group consisting of N, S, and O.

12. A compound according to claim 11 in which X represents methylene, alkylene, or alkylidene substituted with halogen-, cyano-, nitro-, C₁-C₄-alkoxy-, C₁-C₄-halogenoalkoxy-, C₁-C₄-alkylthio-, or C₁-C₄-halogenoalkylthio-substituted C₁-C₄-alkyl, C₂-C₄-alkenyl, or C₂-C₄-alkynyl or substituted with phenyl that is optionally mono- to trisubstituted by identical or different substituents selected from the group consisting of halogen, cyano, nitro, C₁-C₄-alkyl, C₁-C₄-halogenoalkyl, C₁-C₄-alkoxy, C₁-C₄-halogenoalkoxy, C₁-C₄-alkylthio, and C₁-C₄-halogenoalkylthio.

13. A compound according to claim 11 in which X and R¹ are linked by alkylene or alkylidene substituted with optionally halogen-, cyano-, nitro-, C₁-C₄-alkoxy-, C₁-C₄-halogenoalkoxy-, C₁-C₄-alkylthio-, or C₁-C₄-halogenoalkylthio-substituted C₁-C₄-alkyl, C₂-C₄-alkenyl or C₂-C₄-alkynyl, or substituted with phenyl that is optionally mono- to trisubstituted by identical or different substituents selected from the group consisting of halogen, cyano, nitro, C₁-C₄-alkyl, C₁-C₄-halogenoalkyl, C₁-C₄-alkoxy, C₁-C₄-halogenoalkoxy, C₁-C₄-alkylthio, and C₁-C₄-halogenoalkylthio.

14. A compound according to claim 11 in which
X represents substituted methylene or optionally substituted ethylene, propylene or butylene in which the substituents are methyl, ethyl, n- or i-propyl, or n-, i-, s-, or t-butyl, each of which substituents is in turn optionally mono- or poly-substituted by identical or different substituents selected from the group consisting of fluorine, chlorine, methyl, methoxy, and trifluoromethyl,
where two substituents X together with the C atom or the C atoms to which they are attached optionally represent cycloalkyl having 3 to 6 carbon atoms, and
where X and R¹ are optionally linked by optionally substituted alkylene or alkylidene having 1 to 3 carbon atoms in which the substituents are methyl, ethyl, n- or i-propyl, or n-, i-, s-, or t-butyl, each of which substituents is in turn optionally mono- or polysubstituted by identical or different substituents selected from the group consisting of fluorine, chlorine, methyl, methoxy, and trifluoromethyl,
n represents the numbers 1 to 3,
R¹ represents amino, cyano, fluorine, chlorine, bromine, or nitro; represents optionally fluorine- or chlorine-substituted methyl, ethyl, n- or i-propyl, n-, i-, s-, or t-butyl, methoxy, ethoxy, n- or i-propoxy, n-, i-, s- or t-butoxy, methylamino, ethylamino, n- or i-propylamino, or dimethylamino; represents optionally fluorine- and/or chlorine-substituted acetyl, propionyl, methoxycarbonyl, ethoxycarbonyl, n- or i-propoxycarbonyl, methylthio, ethylthio, methylsulphinyl, ethylsulphinyl, methylsulphonyl, ethylsulphonyl, aminocarbonyl, aminothiocarbonyl, methylaminocarbonyl, ethylaminocarbonyl, n- or i-propylaminocarbonyl, dimethylaminocarbonyl, diethylaminocarbonyl, aminosulphonyl, methylaminosulphonyl, ethylaminosulphonyl, dimethylaminosulphonyl, or diethylaminosulphonyl; or represents optionally cyano-, nitro-, fluorine-, chlorine-, methyl-, ethyl-, n- or i-propyl-, n-, i-, s-, or t-butyl-, trifluoromethyl-, methoxy-, ethoxy-, n- or i-propoxy-, methylthio-, ethylthio-, n- or i-propylthio-, methylsulphinyl-, ethylsulphinyl-, n- or i-propylsulphinyl-, methylsulphonyl-, ethylsulphonyl-, n- or i-propylsulphonyl-, difluoromethoxy-, trifluoromethoxy-, trifluoromethylthio-, trifluoromethylsulphinyl-, or trifluoromethylsulphonyl-substituted phenyl, phenoxy, phenylthio, phenylsulphinyl, phenylsulphonyl, benzyl, phenylamino, pyridyloxy, or pyridylamino,
with the proviso that two radicals R¹ in the 3- and 4-positions of the phenyl ring do not both represent optionally fluorine- or chlorine-substituted methoxy, ethoxy, n- or i-propoxy, or n-, i-, s-, or t-butoxy, or optionally cyano-, nitro-, fluorine-, chlorine, methyl, ethyl, n- or i-propyl-, n-, i-, s-, or t-butyl-, trifluoromethyl-, methoxy-, ethoxy-, n- or i-propoxy-, methylthio-, ethylthio-, n- or i-propylthio-, methylsulphinyl-, ethylsulphinyl-, n- or i-propylsulphinyl-, methyl-sulphonyl-ethylsulphonyl-, n- or i-propylsulphonyl-, difluoromethoxy-, trifluoromethoxy-, trifluoromethylthio-, trifluoromethylsulphinyl-, or trifluoromethylsulphonyl-substituted phenyloxy or pyridyloxy,
R² represents a group —CR³₂—CR³₂-A¹-, —CR³₂—CR³₂—CR³₂-A²-, -A³—CR³₂—CR³₂-A⁴-, —CR³₂—CR³₂—NR⁴-A⁵-, —CR³₂—CR³₂—O-A⁶-, —CR³₂—S-A⁷-, or —CR³₂-A⁸—CR³₂—CR³₂—,
where the first-mentioned atom of each such group is attached to the nitrogen in the heterocycle according to formula (I), and
A¹ represents NR⁴, O, CR³₂, S, SO, or SO₂,
A² represents NR⁴, O, CR³₂, S, SO, or SO₂,
A³ represents NR⁴ or O,
A⁴ represents NR⁴, O, or S,
A⁵ represents NR⁴, O, CR³₂, or S,
A⁶ represents NR⁴, CR³₂, or S,
A⁷ represents CR³₂ or S, and
A⁸ represents NR⁴, O, or S,
R³ represents hydrogen or C₁-C₄-alkyl, and
R⁴ represents hydrogen or optionally halogen-, cyano-, or C₁-C₆-alkoxy-substituted alkyl having 1 to 6 carbon atoms.

15. A compound according to claim 11 in which
X represents substituted methylene or optionally substituted ethylene, in which the substituents are methyl or ethyl, each of which substituents is in turn optionally mono- or polysubstituted by identical or different substituents selected from the group consisting of fluorine, chlorine, methyl, methoxy, and trifluoromethyl,
where two substituents X together with the C atom or the C atoms to which they are attached optionally represent cycloalkyl having 3 to 6 carbon atoms, and
where X and R¹ are optionally linked by optionally substituted methylene, 1,2-ethanediyl, 1,3-propanediyl, 1,2-ethenediyl or 1,3-propenediyl, in which the substituents are methyl or ethyl, each of which substituents is in turn optionally mono- or polysubstituted by identical or different substituents selected from the group consisting of fluorine, chlorine, methyl, methoxy, and trifluoromethyl,
n represents the number 1 or 2,
R¹ represents amino, cyano, fluorine, chlorine, bromine, nitro, methyl, ethyl, n- or i-propyl, n-, i-, s- or t-butyl, difluoromethyl, trifluoromethyl, methoxy, ethoxy, n- or i-propoxy, difluoromethoxy, trifluoromethoxy, trifluoromethylthio, methylamino, dimethylamino, acetyl, propionyl, methoxycarbonyl, ethoxycarbonyl, methylthio, methylsulphinyl, methylsulphonyl, methylaminocarbonyl, ethylaminocarbonyl, n- or i-propylaminocarbonyl, dimethylaminocarbonyl, or diethylaminocarbonyl; or represents optionally amino-, cyano-, fluorine-, chlorine-, bromine-, nitro-, methyl-, trifluoromethyl-, methoxy-, trifluoromethoxy-, methylthio-, methylsulphinyl-, methylsulphonyl-, trifluoromethylthio-, trifluoromethylsulphinyl-, or trifluoro-methylsulphonyl-substituted phenyl, phenoxy, phenylthio, phenylsulphinyl, phenylsulphonyl, benzyl, phenylamino, pyridyloxy, or pyridylamino,
with the proviso that two radicals R¹ in the 3- and 4-positions of the phenyl ring do not both represent methoxy, ethoxy, n- or i-propoxy, difluoromethoxy, or trifluoromethoxy, or optionally amino-, cyano-, fluorine-, chlorine-, bromine-, nitro-, methyl-, trifluoromethyl-, methoxy-, trifluoromethoxy-, methylthio-, methylsulphinyl-, methylsulphonyl-, trifluoromethylthio-, trifluoromethylsulphinyl-, or trifluoromethylsulphonyl-substituted phenyloxy or pyridyloxy,
R² represents a group —CH₂—CH₂-A¹-, —CH₂—CH₂—CH₂-A²-, -A³-CH₂—CH₂-A⁴-, —CH₂—CH₂—NR⁴-A⁵-, —CH₂—CH₂—O-A⁶-, —CH₂—CH₂—S-A⁷-, or —CH₂-A⁸-CH₂—CH₂—,
where the first-mentioned atom of each such group is attached to the nitrogen in the heterocycle according to formula (I), and
A¹ represents NR⁴, O, CH₂, or S,
A² represents NR⁴, O, S, or CH₂,
A³ represents NR⁴ or O,
A⁴ represents NR⁴ or S,
A⁵ represents NR⁴, CH₂, or S,
A⁶ represents NR⁴, CH₂, or S,
A⁷ represents S or CH₂, and
A⁸ represents S, O, or NR⁴,
R⁴ represents hydrogen; or represents methyl, ethyl, n- or i-propyl, n-, i-, s-, or t-butyl, each of which is optionally mono- or polysubstituted by fluorine, chlorine, bromine, cyano, methoxy, or ethoxy.

16. A compound according to claim 11 in which X represents one of the groups

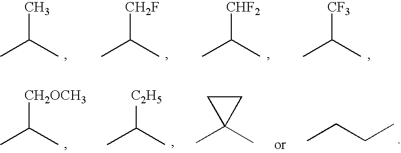

17. A compound of formula (I-c) 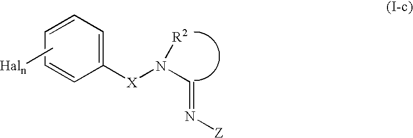
in which
Hal are identical or different and represent F, Cl, or Br,
n represents the numbers 1 to 3,
R² represents a group —CH₂—CH₂-A¹-, —CH₂—CH₂—CH₂-A²-, —CH₂—CH₂—O—CH₂—, or —O—CH₂—CH₂—NR⁴—,
where the first-mentioned atom of each such group is attached to the nitrogen in the heterocycle according to formula (I-c),
A¹ and A² represent NR⁴, O, S, or CH₂,
R⁴ represents hydrogen; or represents methyl, ethyl, n- or i-propyl, n-, i-, s-, or t-butyl, each of which is optionally mono- or polysubstituted by fluorine, chlorine, bromine, cyano, methoxy, or ethoxy,
X represents a group 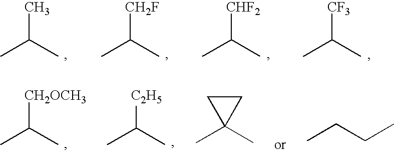
and
Z represents CN or C(S)NH₂.

18. A process for preparing a compound according to claim 11 comprising reacting
(a) a heterocycle of formula (II) 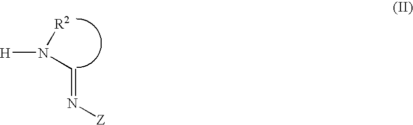
or a salt thereof,
in which
Z represents CN, and
R² has the meanings given for formula (I) in claim 11,
with a substituted phenyl derivative of formula (III) 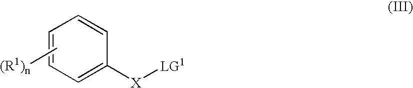
in which
X, R¹, and n have the meanings given for formula (I) in claim 11, and
LG¹ represents a leaving group,
or
(b) a compound of formula (IV) 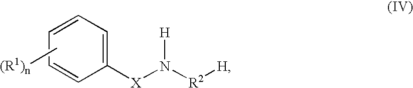
or a salt thereof,
in which X, R¹, R², and n have the meanings given for formula (I) in claim 11,
with the proviso that when R² represents a group —CR³₂—CR³₂-A¹-, —CR³₂—CR³₂—CR³₂-A²-, -A³-CR³₂—CR³₂-A⁴-, —CR³₂—CR³₂—NR⁴-A⁵-, —CR³₂—CR³₂—O-A⁶-, —CR³₂—CR³₂—S-A⁷-, or —CR³₂-A⁸-CR³₂—CR³₂—, where the first-mentioned atom of each such group is attached to the nitrogen in the heterocycle according to formula (I), then
A¹ represents NR⁴, O, S, SO, or SO₂,
A² represents NR⁴, O, S, SO, or SO₂,
A³ represents NR⁴ or O,
A⁴ represents NR⁴, O, or S,
A⁵ represents NR⁴, O, or S,
A⁶ represents NR⁴, or S,
A⁷ represents S, and
A⁸ represents NR⁴, O, or S,
with a compound of formula (V) 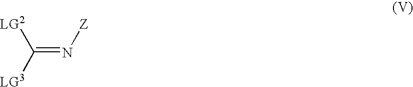
in which
Z represents CN, and
LG² and LG³ represent a leaving group,
or
(c) a heterocycle of formula (VI) 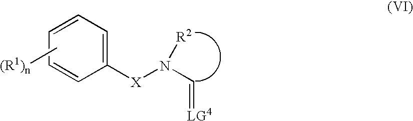
in which
X, R¹, R², and n have the meanings given for formula (I) in claim 11,
LG⁴ represents O or S,
with a compound of formula (VII)
H₂N-Z (VII)
in which Z represents CN or C(S)NH₂,
optionally in the presence of a diluent and optionally in the presence of a basic reaction auxiliary.

19. A process according to claim 18 wherein the heterocycle of formula (II) is used as an alkali metal salt and leaving group LG¹ is halogen, tosylate, or sulphonate.

20. A process according to claim 18 wherein the heterocycle of formula (IV) is used as an alkali metal salt and leaving groups LG² and LG³ are aryloxy or alkyl sulphide.

21. A process according to claim 18 additionally comprising, for compounds in which Z is CN, reacting a compound of formula (Ia) 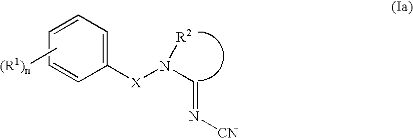
with a sulphur reagent to form a corresponding compound of formula (Ib) 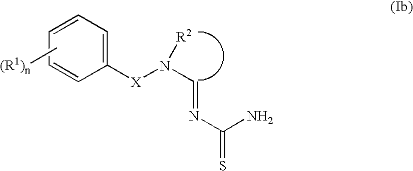
in which X, R¹, R², and n have the meanings given for formula (I) in claim 11.

22. A process according to claim 21 wherein the sulphur reagent is hydrogen sulphide or Lawesson's reagent.

23. A pesticides comprising one or more compounds of formula (I) according to claim 11 and one or more extenders.

24. A method for controlling pests comprising allowing a compound of formula (I) according to claim 11 to act on pests and/or their habitat.
